SEQUENCE LISTING (21 sequences)

SEQ ID NO: 1 - DNA, 2829 bp, Artificial Sequence
  5′ flanking sequence of Genetically Modified Maize event TC1507
actagtttcctagcccgcgtcgtgcccctaccccaccgacgtttatggaaggtgccattccacggttcttcgtggccgcccctaaggatgtaaatggtcggtaaaatccggtaaatttccggtaccgtttaccagatttttccagccgttttcggatttatcgggatatacagaaaacgagacggaaacggaataggttttttttcgaaaacggtacggtaaacggtgagacaaacttaccgtccgttttcgtatttctcgggaaactctggtatattcccgtatttgtcccgtattttcccgacccacggacctgccaatcaaccatcagccagtcagcccatccccacagctatggcccatggggccatgttggccacatgcccacgcaacgcaaggcagtaaggctggcagcctggcacgcattgacgcatgtggacacacacagccgccgcctgttcgtgtttctgtgccgttgtgcgagactgtgactgcgagtggcggagtcggcgaacggcgaggcgtctccggagtctggactgcggctgtggacagcgacgctgtgacggcgactcggcgaagccccaagctaccaagcccccaagtccccatccatctctgcttctctggtcatctccttcccctggtcgatctgcaggcgccagaccggccgaagcatcacgaaacgcactaagacctcgaaggagtcaaaccactcctccgaggcctcgggggctacacccggcgggtgcgctcgcgcgcacccaccggaacaaaatgtaaccgagaaaggtcggtccccttgcaaaaaaagtgcgacaaaagcctccaagcgagtattaacactcactttgaggctcgggggctactgtcggggaccataattaggggtacccccaagactcctaatctcagctggtaacccccatcagcacaaagctgcaaaggcctgatgggtgcgattaagtcaaggctcggtccactcaagggacacgatctcgcctcgcccgagcccagcctcgggcaagggcggccgaccccgaggattcacgtctcgcccgagggccccctcaagcgacgggcacaccttcggctcgcccgaggcccattcttcgccgagaagcaaccttggccagatcgccacaccgaccgaccgtatcgcaggagcatttaatgcgaggatcgcctgacaccttatcctgacgcgcgctcttcagtcgacagagccgaagtgaccgcaatcacttcgccgctccactgaccgacctgacaagaagacagcgccgcctgcgtcgctccgactgctgtgccactcgacagagtgaggctgacagcagccaagtccggcctcgggcgccataggaagctccgcctcgcccgaccctagggctcggactcggcctcggctccggaagacgacgaactacgcttcgcccgaccccagggcttggactcagcctcggctccggaagacgacgaattccgcctcgcccgaccccagggctcggactcggcctcggctccagaagacgacgaactccgcctcgcccgaccccagggctcggactcagcctcggctccggaagacgacgaactccgcctcgcccgaccccagggctcggactcagcctcggcctcagacgatggtctccgcctcgcccgacccggggctcggactcgacctttctatcggaccttgtcagatcctgtcttcgtccgaggaggctttggcaatcctcactatgtactcggtcttaggggagtggcctttcaacaaactggtacgaatcacacccgcacattcaggaactccgggaccattgactctctagatgagataccacctcaagacaacagcggcgcaccttggaatgactactcccatgtgctgaatcatgttacctttgtgcgctggccaggtgagatctcaggttccgactcatggagagcaccaatgttctcttggacgcatcgtagcgctacccccacaaacaccattgatccagagagaatcactcattcttcaagaactgcatatcttgccgagatcctcatccctaaaggtacttgacaatagtattattggagtcgatacacaactcacaaaaaatacaagaagtcgactaggtggattggtccgagtgaagagaaaaaaaagccatacagaactcaaaatcttttccggagatattcattttcctgaagaggcggataagatattaggtggcagtttgataccaccagaaagagaaaaaaaagattctaaggaatcaaaaaaaaggaaaaattgggtttatgttcaacggaaaaaatttctcaaaagcaaggaaaagtattgtggctatttatctatccgtgcagctgatatggccgcggtttgtgatatcgttaaccattacattgagacgtctacagtgaactttaggacagagccacaaacaccacaagagtggattgatgatctagagaggttgcaagatagatacccttggttggttgctgaggttgagggtgttgtggctggtattgcttacgctgggccctggaaggctaggaaccctcaacctcagcaaccaaccaatggtatctatcttgcaacctctctagatcatcaatccactcttgtggtgtttgtggctctgtcctaaagttcactgtagacgtctcaatgtaatggttaacgatatcacaaaccgagagaagagggatctcgaagcttcggccggggcccatcgatatccgcgggcatgcctgcagtgcagcgtgacccggtcgtgcccctctctagagataatgagcattgcatgtctaagttataaaaaattacca

SEQ ID NO: 2 - DNA, 2346 bp, Artificial Sequence
  3′ flanking sequence of Genetically Modified Maize event TC1507
ctcactccgcttgatcttggcaaagatatttgacgcatttattagtatgtgttaattttcatttgcagtgcagtattttctattcgatctttatgtaattcgttacaattaataaatattcaaatcagattattgactgtcatttgtatcaaatcgtgtttaatggatatttttattataatattgatgatatctcaatcaaaacgtagataataataatatttatttaatatttttgcgtcgcacagtgaaaatctatatgagattacaaaataccgacaacattatttaagaaacatagacattaaccctgagactgttggacatcaacgggtagattccttcatgcatagcacctcattcttggggacaaaagcacggtttggccgttccattgctgcacgaacgagctttgctatatcctcgggttggatcatctcatcaggtccaatcaaatttgtccaagaactcatgttagtcgcaacgaaaccggggcatatgtcgggtatctcgagctcgcgaaagcttggctgcaggtcgacggatccttcaacaaaagggtacctgtacccgaaaccgacacaggtgggtaggtagagaatacctaggggcgcgagacaactctctctaaggaactcggcaaaatagccccgtaacttcgggagaaggggtgccccccgctaacaataaacgaatacggtttatgtatggattccggtaaaataccggtactcgatttcataagagtcgaataggaagttaagatgagggtggtatcatcataaaaatggagtagtatcctaaattatactaatccacgtatgatatgtatgcctttccttatcaaccggaagtagtgcaaaaaaaattctatactgcactgctctctttttactgagaaatgcaaaaaaataaaagtgaagtaagggtgccccatagatatttgatcttgcctcctgtcccccccccccttttttcatcaaaaatttccatgaaaaaagaaaagatgaatttgtccattcattgaaccctagttcgggactgacggggctcgaacccgcagcttccgcctgttcctagccttccagggcccagcgtaagcaataccagccacagcaccctcaacctcagcaaccaaccaagggtatctatcttgcaacctctctagatcatcaatccactcttgtggtgtttgtggctctgtcctaaagttcactgtagacgtctcaatgtaatggttaacgatatcacaaaccgcggaacacaagaacgaaagcaccttttcattctttcatatactaggggtttttacttggaaaagacaatgttccatactaaaggatagctgcagaagccgccaccgtcttgaggaccttccggggagccagaccggtcgaaccgtgcctccacttgctaaggagaaagggaaaatcagggccaggacatacgaaggaggagccagaacgaagatatcctaagatacttactcgctccgggccatgatcaatcatgcctgtggggaggtctctcgcacctcgatccatgaaggtaccaccgaggtctgccccgccgccggcttcggtaccgtcctcgccttgggcgcccgaggcacccgggggatggactgcccaggcgcagccacgacgacccaaggatcaccctcctgcgcagtcggcacgagcaatagttctcggggaacaggcagcttggcctgactccccggggtcacctcaactacctcggccgaggggtcaagtaccccctcagtccgcccccgctcttcggaccgggaccccgacgtcccggccccggataccgacggcaccagcccgctcgggggctggcttgacgacccctggcccagcctcagatctgggctgaggccgaggcaggcggccatgtcgtcgtcttcatcatcgtcttcatcatcgtcgtcgtcatcaggcgtctccggcgacggctcccttgggagcccctccctctcctgccgacgacgaagcctttccaaggcatcccgagcccacgtccgctcgtgggcccgagccttctttgcgtccttcttctccttcctcttctccgcggtgaccctccgcgcagctcggtccaccgcatcctccgggactggtggcagggaaggcttgtgatgccctacctcctggagacagacgaaaagtctcagctatgagaaccgagggcaatctgacgcaagaaggaagaaggagcggatactcaccagagacacgcacccgcgatcgggacgcattaagggctgggaaaaagtgccggcctctaatttcgctaccgtgccgtccacccacctgtggaggtcatcgatgggaaggggaa

SEQ ID NO: 3 - DNA, 11361 bp, Artificial Sequence
  Contiguous Sequence for Maize Event TC1507 including 5′ flanking sequence, cry1F insert, and 3′ flanking sequence
actagtttcctagcccgcgtcgtgcccctaccccaccgacgtttatggaaggtgccattccacggttcttcgtggccgcccctaaggatgtaaatggtcggtaaaatccggtaaatttccggtaccgtttaccagatttttccagccgttttcggatttatcgggatatacagaaaacgagacggaaacggaataggttttttttcgaaaacggtacggtaaacggtgagacaaacttaccgtccgttttcgtatttctcgggaaactctggtatattcccgtatttgtcccgtattttcccgacccacggacctgccaatcaaccatcagccagtcagcccatccccacagctatggcccatggggccatgttggccacatgcccacgcaacgcaaggcagtaaggctggcagcctggcacgcattgacgcatgtggacacacacagccgccgcctgttcgtgtttctgtgccgttgtgcgagactgtgactgcgagtggcggagtcggcgaacggcgaggcgtctccggagtctggactgcggctgtggacagcgacgctgtgacggcgactcggcgaagccccaagctaccaagcccccaagtccccatccatctctgcttctctggtcatctccttcccctggtcgatctgcaggcgccagaccggccgaagcatcacgaaacgcactaagacctcgaaggagtcaaaccactcctccgaggcctcgggggctacacccggcgggtgcgctcgcgcgcacccaccggaacaaaatgtaaccgagaaaggtcggtccccttgcaaaaaaagtgcgacaaaagcctccaagcgagtattaacactcactttgaggctcgggggctactgtcggggaccataattaggggtacccccaagactcctaatctcagctggtaacccccatcagcacaaagctgcaaaggcctgatgggtgcgattaagtcaaggctcggtccactcaagggacacgatctcgcctcgcccgagcccagcctcgggcaagggcggccgaccccgaggattcacgtctcgcccgagggccccctcaagcgacgggcacaccttcggctcgcccgaggcccattcttcgccgagaagcaaccttggccagatcgccacaccgaccgaccgtatcgcaggagcatttaatgcgaggatcgcctgacaccttatcctgacgcgcgctcttcagtcgacagagccgaagtgaccgcaatcacttcgccgctccactgaccgacctgacaagaagacagcgccgcctgcgtcgctccgactgctgtgccactcgacagagtgaggctgacagcagccaagtccggcctcgggcgccataggaagctccgcctcgcccgaccctagggctcggactcggcctcggctccggaagacgacgaactacgcttcgcccgaccccagggcttggactcagcctcggctccggaagacgacgaattccgcctcgcccgaccccagggctcggactcggcctcggctccagaagacgacgaactccgcctcgcccgaccccagggctcggactcagcctcggctccggaagacgacgaactccgcctcgcccgaccccagggctcggactcagcctcggcctcagacgatggtctccgcctcgcccgacccggggctcggactcgacctttctatcggaccttgtcagatcctgtcttcgtccgaggaggctttggcaatcctcactatgtactcggtcttaggggagtggcctttcaacaaactggtacgaatcacacccgcacattcaggaactccgggaccattgactctctagatgagataccacctcaagacaacagcggcgcaccttggaatgactactcccatgtgctgaatcatgttacctttgtgcgctggccaggtgagatctcaggttccgactcatggagagcaccaatgttctcttggacgcatcgtagcgctacccccacaaacaccattgatccagagagaatcactcattcttcaagaactgcatatcttgccgagatcctcatccctaaaggtacttgacaatagtattattggagtcgatacacaactcacaaaaaatacaagaagtcgactaggtggattggtccgagtgaagagaaaaaaaagccatacagaactcaaaatcttttccggagatattcattttcctgaagaggcggataagatattaggtggcagtttgataccaccagaaagagaaaaaaaagattctaaggaatcaaaaaaaaggaaaaattgggtttatgttcaacggaaaaaatttctcaaaagcaaggaaaagtattgtggctatttatctatccgtgcagctgatatggccgcggtttgtgatatcgttaaccattacattgagacgtctacagtgaactttaggacagagccacaaacaccacaagagtggattgatgatctagagaggttgcaagatagatacccttggttggttgctgaggttgagggtgttgtggctggtattgcttacgctgggccctggaaggctaggaaccctcaacctcagcaaccaaccaatggtatctatcttgcaacctctctagatcatcaatccactcttgtggtgtttgtggctctgtcctaaagttcactgtagacgtctcaatgtaatggttaacgatatcacaaaccgagagaagagggatctcgaagcttcggccggggcccatcgatatccgcgggcatgcctgcagtgcagcgtgacccggtcgtgcccctctctagagataatgagcattgcatgtctaagttataaaaaattaccacaactggaagagcggttacccggaccgaagcttcggccggggcccatcgatatccgcgggcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcacggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactctagaggatccaacaatggagaacaacatacagaatcagtgcgtcccctacaactgcctcaacaatcctgaagtagagattctcaacgaagagaggtcgactggcagattgccgttagacatctccctgtcccttacacgtttcctgttgtctgagtttgttccaggtgtgggagttgcgtttggcctcttcgacctcatctggggcttcatcactccatctgattggagcctctttcttctccagattgaacagttgattgaacaaaggattgagaccttggaaaggaatcgggccatcactacccttcgtggcttagcagacagctatgagatctacattgaagcactaagagagtgggaagccaatcctaacaatgcccaactgagagaagatgtgcgtatacgctttgctaacacagatgatgctttgatcacagccatcaacaacttcacccttaccagcttcgagatccctcttctctcggtctatgttcaagctgctaacctgcacttgtcactactgcgcgacgctgtgtcgtttgggcaaggttggggactggacatagctactgtcaacaatcactacaacagactcatcaatctgattcatcgatacacgaaacattgtttggatacctacaatcagggattggagaacctgagaggtactaacactcgccaatgggccaggttcaatcagttcaggagagaccttacacttactgtgttagacatagttgctctctttccgaactacgatgttcgtacctatccgattcaaacgtcatcccaacttacaagggagatctacaccagttcagtcattgaagactctccagtttctgcgaacatacccaatggtttcaacagggctgagtttggagtcagaccaccccatctcatggacttcatgaactctttgtttgtgactgcagagactgttagatcccaaactgtgtggggaggacacttagttagctcacgcaacacggctggcaatcgtatcaactttcctagttacggggtcttcaatcccgggggcgccatctggattgcagatgaagatccacgtcctttctatcggaccttgtcagatcctgtcttcgtccgaggaggctttggcaatcctcactatgtactcggtcttaggggagtggcctttcaacaaactggtacgaatcacacccgcacattcaggaactccgggaccattgactctctagatgagataccacctcaagacaacagcggcgcaccttggaatgactactcccatgtgctgaatcatgttacctttgtgcgctggccaggtgagatctcaggttccgactcatggagagcaccaatgttctcttggacgcatcgtagcgctacccccacaaacaccattgatccagagagaatcactcagattcccttggtgaaggcacacacacttcagtcaggaactacagttgtaagagggccggggttcacgggaggagacattcttcgacgcactagtggaggaccattcgcgtacaccattgtcaacatcaatgggcaacttccccaaaggtatcgtgccaggatacgctatgcctctactaccaatctaagaatctacgttacggttgcaggtgaacggatctttgctggtcagttcaacaagacaatggataccggtgatccacttacattccaatctttctcctacgccactatcaacaccgcgttcacctttccaatgagccagagcagtttcacagtaggtgctgataccttcagttcaggcaacgaagtgtacattgacaggtttgagttgattccagttactgccacactcgagtaaggatccgtcgacctgcagccaagctttcgcgagctcgagatccccgacatatgccccggtttcgttgcgactaacatgagttcttggacaaatttgattggacctgatgagatgatccaacccgaggatatagcaaagctcgttcgtgcagcaatggaacggccaaaccgtgcttttgtccccaagaatgaggtgctatgcatgaaggaatctacccgttgatgtccaacagtctcagggttaatgtctatgtatcttaaataatgttgtcggtattttgtaatctcatatagattttcactgtgcgacgcaaaaatattaaataaatattattattatctacgttttgattgagatatcatcaatattataataaaaatatccattaaacacgatttgatacaaatgacagtcaataatctgatttgaatatttattaattgtaacgaattacataaagatcgaatagaaaatactgcactgcaaatgaaaattaacacatactaataaatgcgtcaaatatctttgccaagatcaagcggagtgagggcctcatatccggtctcagttacaagcacggtatccccgaagcgcgctccaccaatgccctcgacatagatgccgggctcgacgctgaggacattgcctaccttgagcatggtctcagcgccggctttaagctcaatcccatcccaatctgaatatcctatcccgcgcccagtccggtgtaagaacgggtctgtccatccacctctgttgggaattccggtccgggtcacctttgtccaccaagatggaactgcggccgcggaccgaattcccatggagtcaaagattcaaatagaggacctaacagaactcgccgtaaagactggcgaacagttcatacagagtctcttacgactcaatgacaagaagaaaatcttcgtcaacatggtggagcacgacacgcttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacagggtacccggggatccaccatgtctccggagaggagaccagttgagattaggccagctacagcagctgatatggccgcggtttgtgatatcgttaaccattacattgagacgtctacagtgaactttaggacagagccacaaacaccacaagagtggattgatgatctagagaggttgcaagatagatacccttggttggttgctgaggttgagggtgttgtggctggtattgcttacgctgggccctggaaggctaggaacgcttacgattggacagttgagagtactgtttacgtgtcacataggcatcaaaggttgggcctaggatccacattgtacacacatttgcttaagtctatggaggcgcaaggttttaagtctgtggttgctgttataggccttccaaacgatccatctgttaggttgcatgaggctttgggatacacagcccggggtacattgcgcgcagctggatacaagcatggtggatggcatgatgttggtttttggcaaagggattttgagttgccagctcctccaaggccagttaggccagttacccagatctgagtcgacctgcaggcatgcccgctgaaatcaccagtctctctctacaaatctatctctctctataataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtggcgagctcgaattcgagctcgagcccgggtggatcctctagagtcgacctgcagaagcttcggtccggcgcgcctctagttgaagacacgttcatgtcttcatcgtaagaagacactcagtagtcttcggccagaatggcctaactcaaggccctcactccgcttgatcttggcaaagatatttgacgcatttattagtatgtgttaattttcatttgcagtgcagtattttctattcgatctttatgtaattcgttacaattaataaatattcaaatcagattattgactgtcatttgtatcaaatcgtgtttaatggatatttttattataatattgatgatatctcaatcaaaacgtagataataataatatttatttaatatttttgcgtcgcacagtgaaaatctatatgagattacaaaataccgacaacattatttaagaaacatagacattaaccctgagactgttggacatcaacgggtagattccttcatgcatagcacctcattcttggggacaaaagcacggtttggccgttccattgctgcacgaacgagctttgctatatcctcgggttggatcatctcatcaggtccaatcaaatttgtccaagaactcatgttagtcgcaacgaaaccggggcatatgtcgggtatctcgagctcgcgaaagcttggctgcaggtcgacggatccttcaacaaaagggtacctgtacccgaaaccgacacaggtgggtaggtagagaatacctaggggcgcgagacaactctctctaaggaactcggcaaaatagccccgtaacttcgggagaaggggtgccccccgctaacaataaacgaatacggtttatgtatggattccggtaaaataccggtactcgatttcataagagtcgaataggaagttaagatgagggtggtatcatcataaaaatggagtagtatcctaaattatactaatccacgtatgatatgtatgcctttccttatcaaccggaagtagtgcaaaaaaaattctatactgcactgctctctttttactgagaaatgcaaaaaaataaaagtgaagtaagggtgccccatagatatttgatcttgcctcctgtcccccccccccttttttcatcaaaaatttccatgaaaaaagaaaagatgaatttgtccattcattgaaccctagttcgggactgacggggctcgaacccgcagcttccgcctgttcctagccttccagggcccagcgtaagcaataccagccacagcaccctcaacctcagcaaccaaccaagggtatctatcttgcaacctctctagatcatcaatccactcttgtggtgtttgtggctctgtcctaaagttcactgtagacgtctcaatgtaatggttaacgatatcacaaaccgcggaacacaagaacgaaagcaccttttcattctttcatatactaggggtttttacttggaaaagacaatgttccatactaaaggatagctgcagaagccgccaccgtcttgaggaccttccggggagccagaccggtcgaaccgtgcctccacttgctaaggagaaagggaaaatcagggccaggacatacgaaggaggagccagaacgaagatatcctaagatacttactcgctccgggccatgatcaatcatgcctgtggggaggtctctcgcacctcgatccatgaaggtaccaccgaggtctgccccgccgccggcttcggtaccgtcctcgccttgggcgcccgaggcacccgggggatggactgcccaggcgcagccacgacgacccaaggatcaccctcctgcgcagtcggcacgagcaatagttctcggggaacaggcagcttggcctgactccccggggtcacctcaactacctcggccgaggggtcaagtaccccctcagtccgcccccgctcttcggaccgggaccccgacgtcccggccccggataccgacggcaccagcccgctcgggggctggcttgacgacccctggcccagcctcagatctgggctgaggccgaggcaggcggccatgtcgtcgtcttcatcatcgtcttcatcatcgtcgtcgtcatcaggcgtctccggcgacggctcccttgggagcccctccctctcctgccgacgacgaagcctttccaaggcatcccgagcccacgtccgctcgtgggcccgagccttctttgcgtccttcttctccttcctcttctccgcggtgaccctccgcgcagctcggtccaccgcatcctccgggactggtggcagggaaggcttgtgatgccctacctcctggagacagacgaaaagtctcagctatgagaaccgagggcaatctgacgcaagaaggaagaaggagcggatactcaccagagacacgcacccgcgatcgggacgcattaagggctgggaaaaagtgccggcctctaatttcgctaccgtgccgtccacccacctgtggaggtcatcgatgggaaggggaa

SEQ ID NO: 4 - DNA, 19 bp, Artificial Sequence
  primer
tagtcttcggccagaatgg

SEQ ID NO: 5 - DNA, 18 bp, Artificial Sequence
  primer
ctttgccaagatcaagcg

SEQ ID NO: 6 - DNA, 21 bp, Artificial Sequence
  probe
taactcaaggccctcactccg

SEQ ID NO: 7 - DNA, 18 bp, Artificial Sequence
  primer
cgctctgtacaagcgtgc

SEQ ID NO: 8 - DNA, 21 bp, Artificial Sequence
  primer
gcaaagtgttgtgcttggacc

SEQ ID NO: 9 - DNA, 29 bp, Artificial Sequence
  probe
cacgtgagaatttccgtctactcgagcct

SEQ ID NO: 10 - DNA, 18 bp, Artificial Sequence
  primer
tggcggacgacgacttgt

SEQ ID NO: 11 - DNA, 19 bp, Artificial Sequence
  primer
aaagtttggaggctgccgt

SEQ ID NO: 12 - DNA, 26 bp, Artificial Sequence
  probe
cgagcagaccgccgtgtacttctacc

SEQ ID NO: 13 - DNA, 23 bp, Artificial Sequence
  primer
cgtcgtttcccatctcttcctcc

SEQ ID NO: 14 - DNA, 20 bp, Artificial Sequence
  primer
ccactccgagaccctcagtc

SEQ ID NO: 15 - DNA, 27 bp, Artificial Sequence
  probe
aatcagggctcattttctcgctcctca

SEQ ID NO: 16 - DNA, 23 bp, Artificial Sequence
  primer
ttggactagaaatctcgtgctga

SEQ ID NO: 17 - DNA, 22 bp, Artificial Sequence
  primer
gctacatagggagccttgtcct

SEQ ID NO: 18 - DNA, 23 bp, Artificial Sequence
  probe
caatccacacaaacgcacgcgta

SEQ ID NO: 19 - DNA, 20 bp, Artificial Sequence
  primer
tgcagcaactgttggcctta

SEQ ID NO: 20 - DNA, 23 bp, Artificial Sequence
  primer
tcatgttaggcgtcatcatctgt

SEQ ID NO: 21 - DNA, 24 bp, Artificial Sequence
  probe
catcactggcatcgtctgaagcgg

(19) United States
(12) Patent Application Publication
(10) Pub. No.: US 2011/0151441 A1
(43) Pub. Date: Jun. 23, 2011

(54) ENDPOINT TAQMAN METHODS FOR DETERMINING ZYGOSITY OF CORN COMPRISING TC1507 EVENTS

(75) Inventors: Wei Chen, Carmel, IN (US); Wesley Marchione, Greenfield, IN (US); Stephen Novak, Westfield, IN (US); Manju Gupta, Carmel, IN (US); Thomas W. Greene, Zionsville, IN (US); Siva Kumptla, Carmel, IN (US)

(73) Assignee: Dow AgroSciences LLC, Indianapolis, IN (US)

(21) Appl. No.: 12/642,352

(22) Filed: Dec. 18, 2009

(51) Int. Cl. C12Q 1/68

(52) U.S. Cl. 435/6

(57) ABSTRACT

A method for zygosity analysis of the maize Cry1F event TC1507 is provided. The method provides TC1507 event-specific and maize endogenous reference gene-specific primers and TaqMan probe combinations for use in an endpoint biplex TaqMan PCR assay capable of producing robust genotype calls for assisting in molecular breeding of TC1507.

### BACKGROUND OF THE INVENTION

Herculex® I is a commercial maize product, comprising Cry1F event TC1507, which is resistant to insect damage (particularly by European corn borer). The event, itself, is disclosed in, for example, U.S. Pat. Nos. 7,605,310 and 7,449,564.

Various methods can be used to detect the presence of this event in a sample of corn. One example is the Pyrosequencing technique as described by Winge (Innov. Pharma. Tech. 00:18-24, 2000). In this method an oligonucleotide is designed that overlaps the adjacent genomic DNA and insert DNA junction. The oligonucleotide is hybridized to single-stranded PCR product from the region of interest (one primer in the inserted sequence and one in the flanking genomic sequence) and incubated in the presence of a DNA polymerase, ATP, sulfurylase, luciferase, apyrase, adenosine 5′ phosphosulfate and luciferin. DNTPs are added individually and the incorporation results in a light signal that is measured. A light signal indicates the presence of the transgene insert/flanking sequence due to successful amplification, hybridization, and single or multi-base extension. (This technique is usually used for initial sequencing, not for detection of a specific gene when it is known.)

Fluorescence Polarization is another method that can be used to detect an amplicon. Following this method, an oligonucleotide is designed to overlap the genomic flanking and inserted DNA junction. The oligonucleotide is hybridized to single-stranded PCR product from the region of interest (one primer in the inserted DNA and one in the flanking genomic DNA sequence) and incubated in the presence of a DNA polymerase and a fluorescent-labeled ddNTP. Single base extension results in incorporation of the ddNTP. Incorporation can be measured as a change in polarization using a fluorometer. A change in polarization indicates the presence of the transgene insert/flanking sequence due to successful amplification, hybridization, and single base extension.

TAQMAN (Life Technologies, Foster City, Calif.) is a method of detecting and quantifying the presence of a DNA sequence. Briefly, a FRET oligonucleotide probe is designed with one oligo within the transgene and one in the flanking genomic sequence for event-specific detection. The FRET probe and PCR primers (one primer in the insert DNA sequence and one in the flanking genomic sequence) are cycled in the presence of a thermostable polymerase and dNTPs. Hybridization of the FRET probe results in cleavage and release of the fluorescent moiety away from the quenching moiety on the FRET probe. A fluorescent signal indicates the presence of the flanking/transgene insert sequence due to successful amplification and hybridization.

Molecular Beacons have been described for use in sequence detection. Briefly, a FRET oligonucleotide probe is designed that overlaps the flanking genomic and insert DNA junction. The unique structure of the FRET probe results in it containing secondary structure that keeps the fluorescent and quenching moieties in close proximity. The FRET probe and PCR primers (one primer in the insert DNA sequence and one in the flanking genomic sequence) are cycled in the presence of a thermostable polymerase and dNTPs. Following successful PCR amplification, hybridization of the FRET probe to the target sequence results in the removal of the probe secondary structure and spatial separation of the fluorescent and quenching moieties. A fluorescent signal indicates the presence of the flanking genomic/transgene insert sequence due to successful amplification and hybridization.

Another challenge, among many, is finding a suitable reference gene for a given test. For example, as stated in the abstract of Czechowski et al., “An exceptionally large set of data from Affymetrix ATH1 whole-genome GeneChip studies provided the means to identify a new generation of reference genes with very stable expression levels in the model plant species Arabidopsis (Arabidopsis thaliana). Hundreds of Arabidopsis genes were found that outperform traditional reference genes in terms of expression stability throughout development and under a range of environmental conditions.” (Czechowski et al. (2005) Genome-wide identification and testing of superior reference genes for transcript normalization in Arabidopsis. Plant Physiol. 139, 5-17.)

Brodmann et al. (2002) relates to real-time quantitative PCR detection of transgenic maize content in food for four different maize varieties approved in the European Union. Brodmann, P.D., P.D., Ilg E. C., Berthoud H., and Herrmann, A. Real-Time Quantitative Polymerase Chain Reaction Methods for Four Genetically Modified Maize Varieties and Maize DNA Content in Food. J. of AOAC international 2002 85 (3)

Hernandez et al. (2004) mentions four possible genes for use with real-time PCR. Hernandez, M., Duplan, M.-N., Berthier, G., Vaitilingom, M., Hauser, W., Freyer, R., Pla, M., and Bertheau, Y. Development and comparison of four real-time polymerase chain reation systems for specific detection and quantification of Zea mays L. J. Agric. Food Chem. 2004, 52, 4632-4637.

Costa et al. (2007) looked at these four genes (also in the real-time PCR context) and concluded that the alcohol dehydrogenase and zein genes were the best reference genes for detecting a sample “event” (a lectin gene) for transgenic feed intermix issues. Costa, L. D., and Martinelli L. Development of a Real-Time PCR Method Based on Duplo Target Plasmids for Determining an Unexpected Genetically Modified Soybean Intermix with Feed Components. J. Agric. Food Chem. 2007, 55, 1264-1273.

Huang et al. (2004) used plasmid pMulM2 as reference molecules for detection of MON810 and NK603 transgenes in maize. Huang and Pan, “Detection of Genetically Modified Maize MON810 and NK603 by Multiplex and Real-Time Polymerase Chain Reaction Methods,” J. Agric. Food Chem., 2004, 52 (11), pp 3264-3268.

Gasparic et al. (2008) suggest LNA technology, from a comparison to cycling probe technology, TaqMan, and various real-time PCR chemistries, for quantitatively analyzing maize events (such as MON810). Ga\check{s}pari\check{c}, Cankar, \check{Z}el, and Gruden, “Comparison of different real-time PCR chemistries and their suitability for detection and quantification of genetically modified organisms,” BMC Biotechnol. 2008; 8: 26.

US 20070148646 relates to a primer extension method for quantification that requires controlled dispensation of individual nucleotides that can be detected and quantified by the amount of nucleotides incorporated. This is different from the TaqMan PCR method using an internal reference gene.

To distinguish between homozygous and hemizygous genotypes of TC1507, an Invader assay has been successfully used for this event. Gupta, M., Nirunsuksiri, W., Schulenberg, G., Hartl, T., Novak, S., Bryan, J., Vanopdorp, N., Bing, J. and Thompson, S. A non-PCR-based Invader Assay Quantitatively Detects Single-Copy Genes in Complex Plant Genomes. Mol. Breeding 2008, 21, 173-181.

Huabang (2009) relates to PCR-based zygosity testing of transgenic maize. However, no reference gene appears to be used. Huabang, “An Accurate and Rapid PCR-Based Zygosity Testing Method for Genetically Modified Maize,” Molecular Plant Breeding, 2009, Vol. 7, No. 3, 619-623.

### BRIEF SUMMARY OF THE INVENTION

The present invention relates in part to a molecular assay for determining zygosity of event TC1507 in maize. More specifically, the present invention relates in part to an endpoint TaqMan PCR assay for a Herculex® I event TC1507 in corn utilizing a maize endogenous reference gene. Some embodiments are directed to assays that are capable of high throughput zygosity analysis. The subject invention further relates, in part, to the discovery of a preferred reference gene for use in determining zygosity.

### BRIEF DESCRIPTION OF THE DRAWINGS

FIG. 1 shows an example of a distribution graph between sample numbers and absolute ratios of SOB1/SOB2.

FIG. 2 is real-time PCR amplification plots of biplex combination of TC1507 with different reference genes investigated. Real-time PCR amplification plots are shown for biplex of TC1507 with ivr (2a), hmg (2b), ivr104(2c), and zein (2d) with 2 fold serial dilutions of Cry1F homozygotes genomic DNA, respectively. Ct values with each dilution are shown inside their corresponding plots.

FIG. 3 is distribution graphs of Cry1F zygosity determinations with endpoint TaqMan using different reference genes. Panels are as follows, for assays using: ivr104 (a), ivr (b), hmg (c) and zein (d) as reference genes. On completion of PCR and fluorescence readings, distribution graphs were generated: SOB1=Signal over the background of FAM (sample signal over average of background signal at 535 nm), SOB2=Signal over background of Cy5 (sample signal over average of background signal at 670 nm). For ivr104 (a), genotype calls can be made with SOB1/SOB2<0.5 for wild type, 0.5<SOB1/SOB2<2 for hemizygotes, and SOB1/SOB2>2 for homozygotes.

FIG. 4 shows validation of Cry1F zygosity determination with endpoint TaqMan assay using ivr104 as reference gene on three populations (two 96 well plates of genomic DNA for each population). On completion of PCR and fluorescence readings, a distribution graph was generated: SOB1=Signal over the background of FAM (ratio of sample signal over average of background signal at 535 nm), SOB2=Signal over background of Cy5 (Ratio of sample signal over average of background signal at 670 nm). Genotype calls were made accordingly with distinct clusters of homozygotes, hemizygotes and wild types. FIG. 4a is a distribution graph of Cry1F zygosity determination with endpoint TaqMan assay on a Cry34/35 PoCry1F stack population. FIG. 4b is a distribution graph of Cry1F zygosity determination with endpoint TaqMan assay on a PoCry1F single stack population. FIG. 4c (PoCry1F_NK603) had only two clusters (homozygotes and hemizygotes) since the WT plants, as expected, did not survive the herbicide spray.

### BRIEF DESCRIPTION OF THE SEQUENCES

SEQ ID NO:1 is a sequence of the 5′ flanking sequence of maize event TC1507.

SEQ ID NO:2 is a sequence of the 3′ flanking sequence of maize event TC1507.

SEQ ID NO:3 is a contiguous sequence for maize event TC1507 including a 5′ flanking sequence, the cry1F insert, and a 3′ flanking sequence.

SEQ ID NOs:4-21 are exemplified primers and probes for use according to the subject invention.

### DETAILED DESCRIPTION OF THE INVENTION

The subject invention relates in part to a fluorescence-based endpoint TaqMan PCR assay utilizing an endogenous gene as a reference (copy number) control for high-throughput zygosity analysis of TC1507, a maize Cry1F event. The subject invention further relates, in part, to the discovery of a preferred reference gene, invertase. Several reference genes were identified as possible options.

The subject invention also relates in part to the development of a biplex endpoint TaqMan PCR for TC1507 event specific zygosity analysis. Further, the subject invention relates in part to the development of TC1507 breeding test kits.

Endpoint TaqMan assays are based on a plus/minus strategy, by which a “plus” signifies the sample is positive for the assayed gene and a “minus” signifies the sample is negative for the assayed gene. These assays typically utilize two sets of oligonucleotides for identifying the TC1507 transgene sequence and the wild-type gene sequence respectively, as well as dual-labeled probes to measure the content of transgene and wild type sequence.

Although the Invader assay has been a robust technique for characterizing these events, it is very sensitive to DNA quality. In addition, the assay requires a high quantity of DNA. Invader also requires an additional denaturing step which, if not handled properly, can render the Invader assay unsuccessful. Additionally, the longer assay time of the Invader assay is limited in its flexibility to efficiently handle large numbers of TC1507 samples for analysis in a commercial setting. One main advantage of the subject invention is time savings and elimination of the denaturing step.

The subject Endpoint TaqMan analysis for detecting TC1507 events offers surprising advantages over Invader, particularly in analyzing large number of samples. However, its application to TC1507 was complicated. For one, the border region of TC1507 is not the true genomic sequence. For example, it contains repetitive flanking sequences. It is a unique sequence containing transgene fragments and retrotransposons, which would have been perceived as a very significant obstacle to the implementation of the endpoint Taqman assay plus/minus strategy in this context.

In addition, for example, multiple primer and probe combinations were tried. However, none of those combinations resulted in a robust signal capable of discriminating between the wild-type allele and the transgene.

In one embodiment, the TC1507 event-specific PCR reaction amplifies a 58-bp fragment, unique to the event, resulting from the insertion of the TC1507 construct cassette into the corn genomic DNA. A TC1507 target-specific oligonucleotide probe binds to the target between two event-specific PCR primers and is labeled with a fluorescent dye and quencher. Possible fluorescent labels include FAM as a reporter dye at the TC1507 probe 5′ end and a Black Hole Quencher 1 (BHQ1) as the quencher at the TC1507 probe 3′ end.

Using a range of empirical factors together with our judgment, we empirically identified maize endogenous genes, capable of single or low copy number PCR amplification, and which prove to be conserved in many cultivars. Many, many reference genes were possibilities. The ones we selected for initial assessment were assessed as possible reference genes for the TC1507 zygosity analysis. Five sets of oligos (Table 1) were selected: ivr, ivr104 (79 and 104 by fragments from invertase), adh (136 by fragment from adh1), hmg (79 bp fragment from hmga), and zein (72 bp fragment from zein). Gene specific primers and a gene-specific probe, in one embodiment labeled with Cy5 at the probe 5′ end and a Black Hole Quencher 2 (BHQ2) at the probe 3′ end, were assessed for use in rapid quantification for the maize endogenous genes assessed as possible reference genes for the TC1507 zygosity analysis.

The gene-specific primers and probes for the Cry1F gene and the maize endogenous genes were tested for PCR efficiencies. Primer and probe combinations of the maize endogenous genes determined to have PCR efficiencies relatively similar to the Cry1F event specific primer and probes were further exploited for multiplexing capabilities and endpoint TaqMan zygosity assay.

All oligos were tested for PCR efficiencies. Oligos with PCR efficiencies relatively close to the event specific oligos were further exploited for multiplexing and endoint TaqMan zygosity assay.

In some embodiments, this zygosity assay utilizes a biplex of oligonucleotides specific to the TC1507 event and to the maize endogenous reference gene (invertase in some preferred embodiments) in the same amplification assay. Zygosity is determined by the relative intensity of fluorescence specific for Event TC1507 as compared to the reference DNA.

In some embodiments, the TC1507 event-specific assay amplifies a 58-bp fragment, unique to the event, resulting from the insertion of the TC1507 construct cassette into the corn genomic DNA. A target-specific oligonucleotide probe binds to the target between two event-specific TC1507 PCR primers and is labeled with two fluorescent dyes: FAM as a reporter dye at its 5′ end and BHQ as a quencher dye at its 3′ end. PCR products are measured after optimal number of cycles, when the reaction is in the early exponential phase.

In some embodiments, the maize-specific reference system amplifies a 104 bp fragment of the invertase gene. A pair of invertase specific oligos and an invertase gene-specific probe labeled with Cy5 at the 3′ end and a BHQ at the 5′ end are used for rapid quantitation.

In some embodiments, the fluorescence-based end-point TaqMan assay for TC1507 zygosity analysis allows the results to be directly read in a plate reader for identification of the Herculex® I Event TC1507 in corn and the reference gene.

The subject invention includes breeding applications such as testing the introgression of Herculex® I into other corn lines.

Detection methods and kits of the subject invention can be used to identify events according to the subject invention. Methods and kits of the subject invention can be used for accelerated breeding strategies and to establish linkage data. Detection techniques of the subject invention are especially useful in conjunction with plant breeding, to determine which progeny plants comprise a given event, after a parent plant comprising an event of interest is crossed with another plant line in an effort to impart one or more additional traits of interest in the progeny. These Taqman PCR analysis methods benefit maize breeding programs as well as quality control, especially for commercialized transgenic maize seeds. Taqman PCR detection kits for these transgenic maize lines can also now be made and used. This can also benefit product registration and product stewardship.

Still further, the subject invention can be used to study and characterize transgene integration processes, genomic integration site characteristics, event sorting, stability of transgenes and their flanking sequences, and gene expression (especially related to gene silencing, transgene methylation patterns, position effects, and potential expression-related elements such as MARS [matrix attachment regions], and the like).

This invention further includes processes of making crosses using a TC1507 plant as at least one parent. For example, the subject invention includes an F₁ hybrid plant having as one or both parents any of the plants exemplified herein. This invention includes a method for producing an F₁ hybrid seed by crossing an exemplified plant with a different (e.g. in-bred parent) plant, harvesting the resultant hybrid seed, and testing the seed/plant sample according to the subject invention. Characteristics of the resulting plants may be improved by careful consideration of the parent plants.

An insect-resistant maize plant can be bred by first sexually crossing a first parental maize plant consisting of a maize plant grown from seed of any one of the lines referred to herein, and a second parental maize plant, thereby producing a plurality of first progeny plants; and then selecting a first progeny plant that is resistant to insects (or that possesses at least one of the events of the subject invention); and selfing the first progeny plant, thereby producing a plurality of second progeny plants; and then selecting from the second progeny plants a plant that is resistant to insects (or that possesses at least one of the events of the subject invention). These steps can further include the back-crossing of the first progeny plant or the second progeny plant to the second parental maize plant or a third parental maize plant. A maize crop comprising maize seeds of the subject invention, or progeny thereof, can then be planted.

It is also to be understood that two different transgenic plants can also be mated to produce offspring that contain two independently segregating added, exogenous genes. Selfing of appropriate progeny can produce plants that are homozygous for both added, exogenous genes. Back-crossing to a parental plant and out-crossing with a non-transgenic plant are also contemplated, as is vegetative propagation. Other breeding methods commonly used for different traits and crops are known in the art. Backcross breeding has been used to transfer genes for a simply inherited, highly heritable trait into a desirable homozygous cultivar or inbred line, which is the recurrent parent. The source of the trait to be transferred is called the donor parent. The resulting plant is expected to have the attributes of the recurrent parent (e.g., cultivar) and the desirable trait transferred from the donor parent. After the initial cross, individuals possessing the phenotype of the donor parent are selected and repeatedly crossed (backcrossed) to the recurrent parent. The resulting parent is expected to have the attributes of the recurrent parent (e.g., cultivar) and the desirable trait transferred from the donor parent.

DNA molecules of the present invention can be used as molecular markers in a marker assisted breeding (MAB) method. DNA molecules of the present invention can be used in methods (such as, AFLP markers, RFLP markers, RAPD markers, SNPs, and SSRs) that identify genetically linked agronomically useful traits, as is known in the art. The insect-resistance trait can be tracked in the progeny of a cross with a maize plant of the subject invention (or progeny thereof and any other maize cultivar or variety) using the MAB methods. The DNA molecules are markers for this trait, and MAB methods that are well-known in the art can be used to track the insect-resistance trait(s) in maize plants where at least one maize line of the subject invention, or progeny thereof, was a parent or ancestor. The methods of the present invention can be used to identify any maize variety having the insect-resistance event from maize line TC1507.

Methods of the subject invention include a method of producing an insect-resistant maize plant wherein said method comprises breeding with a plant of the subject invention. More specifically, said methods can comprise crossing two plants of the subject invention, or one plant of the subject invention and any other plant, and tracking the subject event according to the subject invention. Preferred methods further comprise selecting progeny of said cross by analyzing said progeny for an event detectable according to the subject invention.

A preferred plant, or a seed, propagated and developed according to the subject invention comprises in its genome at least one of the insert sequences, as identified in Table 1, together with at least 20-500 or more contiguous flanking nucleotides on both sides of the insert, as identified in Table 1. Unless indicated otherwise, “event TC1507” or like reference refers to DNA of SEQ ID NO:3 that includes the heterologous DNA inserted in the genomic location identified by all or part of both of the flanking genomic sequences of SEQ ID NOs:1 and/or SEQ ID NO:2 immediately adjacent to the inserted DNA that would be expected to be transferred to progeny that receives the inserted DNA as a result of a sexual cross of a parental line that includes the event.

Definitions and examples are provided herein to help describe the present invention and to guide those of ordinary skill in the art to practice the invention. Unless otherwise noted, terms are to be understood according to conventional usage by those of ordinary skill in the relevant art. The nomenclature for DNA bases as set forth at 37 CFR §1.822 is used.

A transgenic “event” is produced by transformation of plant cells with heterologous DNA, i.e., a nucleic acid construct that includes a transgene of interest, regeneration of a population of plants resulting from the insertion of the transgene into the genome of the plant, and selection of a particular plant characterized by insertion into a particular genome location. The term “event” refers to the original transformant and progeny of the transformant that include the heterologous DNA. The term “event” also refers to progeny produced by a sexual outcross between the transformant and another variety that includes the genomic/transgene DNA. Even after repeated back-crossing to a recurrent parent, the inserted transgene DNA and flanking genomic DNA (genomic/transgene DNA) from the transformed parent is present in the progeny of the cross at the same chromosomal location. The term “event” also refers to DNA from the original transformant and progeny thereof comprising the inserted DNA and flanking genomic sequence immediately adjacent to the inserted DNA that would be expected to be transferred to a progeny that receives inserted DNA including the transgene of interest as the result of a sexual cross of one parental line that includes the inserted DNA (e.g., the original transformant and progeny resulting from selfing) and a parental line that does not contain the inserted DNA.

A “junction sequence” spans the point at which DNA inserted into the genome is linked to DNA from the maize native genome flanking the insertion point, the identification or detection of one or the other junction sequences in a plant's genetic material being sufficient to be diagnostic for the event. Included are the DNA sequences that span the insertions in herein-described maize events and similar lengths of flanking DNA. Specific examples of such diagnostic sequences are provided herein; however, other sequences that overlap the junctions of the insertions, or the junctions of the insertions and the genomic sequence, are also diagnostic and could be used according to the subject invention.

Primers, amplicons, and probes can be designed for use according to the subject invention based in part on the flanking, junction, and/or insert sequences. Related primers and amplicons can be included as components of the invention. PCR analysis methods using amplicons that span across inserted DNA and its borders can be used to detect or identify commercialized transgenic maize varieties or lines derived from the subject proprietary transgenic maize lines.

The sequence of the 5′ flanking sequence for HERCULEX I (TC1507 event) is provided as SEQ ID NO:1. The sequence of the 3′ flanking sequence is provided as SEQ ID NO:2. The sequence of the cry1F insert (together with regulatory sequences), flanked by the flanking sequences (of SEQ ID NOs:1 and 2) is provided as SEQ ID NO:3. Table 1 provides the coordinates of the insert and flanking sequences with respect to SEQ ID NO:3.

TABLE 1

| | Residue location in SEQ ID NO: 3: | | |
|---|---|---|---|
| Event | 5′ Flanking | cry1F Insert | 3′Flanking |
| TC1507 | 1-2829 | 2830-9015 | 9016-11361 |
| | (see SEQ ID NO: 1) | | (see SEQ ID NO: 2) |

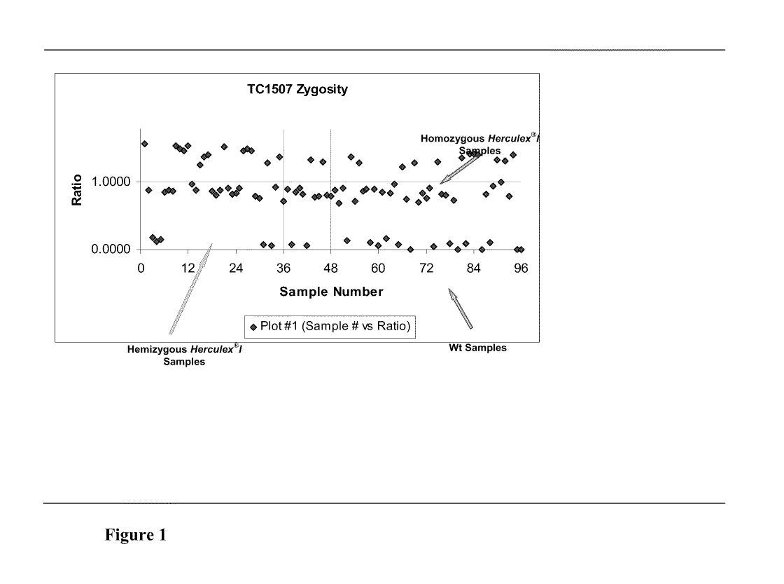

These insertion events, and further components thereof, are further illustrated in, for example, U.S. Pat. Nos. 7,605,310 and 7,449,564 (see e.g. FIG. 1 of the '564 patent). Based on these insert and border sequences, event-specific primers were, and can be, generated. PCR analysis demonstrated that these maize lines can be identified in different maize genotypes by analysis of the PCR amplicons generated with these event-specific primer sets. Thus, these and other related procedures can be used to uniquely identify these maize lines.

As used herein, a “line” is a group of plants that display little or no genetic variation between individuals for at least one trait. Such lines may be created by several generations of self-pollination and selection, or vegetative propagation from a single parent using tissue or cell culture techniques.

As used herein, the terms “cultivar” and “variety” are synonymous and refer to a line which is used for commercial production. “Stability” or “stable” means that with respect to the given component, the component is maintained from generation to generation and, preferably, at least three generations at substantially the same level, e.g., preferably ±15%, more preferably ±10%, most preferably ±5%. The stability may be affected by temperature, location, stress and the time of planting. Comparison of subsequent generations under field conditions should produce the component in a similar manner.

“Commercial Utility” is defined as having good plant vigor and high fertility, such that the crop can be produced by farmers using conventional farming equipment, and the oil with the described components can be extracted from the seed using conventional crushing and extraction equipment. To be commercially useful, the yield, as measured by seed weight, oil content, and total oil produced per acre, is within 15% of the average yield of an otherwise comparable commercial canola variety without the premium value traits grown in the same region.

“Agronomically elite” means that a line has desirable agronomic characteristics such as yield, maturity, disease resistance, and the like, in addition to the insect resistance due to the subject event(s).

As one skilled in the art will recognize in light of this disclosure, preferred embodiments of detection kits, for example, can include probes and/or primers directed to and/or comprising “junction sequences” or “transition sequences” (where the maize genomic flanking sequence meets the insert sequence). For example, this includes a polynucleotide probe, primer, or amplicon comprising a sequence including residues, as indicated in Table 1. Some preferred primers can include at least ˜15 residues of the adjacent flanking sequence and at least ˜15 residues of the adjacent insert sequence. Residues within 200 bases or so of the junction sequences can be targeted. With this arrangement, another primer in either the flanking or insert region can be used to generate a detectable amplicon that indicates the presence of an event of the subject invention. In some preferred embodiments, one primer binds in the flanking region and one binds in the insert, and these primers can be used to generate an amplicon that spans (and includes) a junction sequence (residues 2829-2030 and/or 9015-9016) as indicated above. SEQ ID NOs:1 and/or 2 can be aligned with SEQ ID NO:3 to illustrate such junctions.

One skilled in the art will also recognize that primers and probes can be designed to hybridize, under a range of standard hybridization and/or PCR conditions, to a segment of SEQ ID NO:1, SEQ ID NO:2, SEQ ID NO:3, and complements thereof, wherein the primer or probe is not perfectly complementary to the exemplified sequence. That is, some degree of mismatch can be tolerated. For an approximately 20 nucleotide primer, for example, typically one or two or so nucleotides do not need to bind with the opposite strand if the mismatched base is internal or on the end of the primer that is opposite the amplicon. Various appropriate hybridization conditions are provided below. Synthetic nucleotide analogs, such as inosine, can also be used in probes. Peptide nucleic acid (PNA) probes, as well as DNA and RNA probes, can also be used. What is important is that such probes and primers are diagnostic for (able to uniquely identify and distinguish) the presence of an event of the subject invention.

Components of the transgene “insert” or construct are disclosed in, for example, U.S. Pat. Nos. 7,605,310 and 7,449,564 (see e.g. FIG. 1 of the '564 patent). Polynucleotide sequences or fragments of these components can be used as DNA primers or probes in the methods of the present invention.

In some embodiments of the invention, compositions and methods are provided for detecting the number of copies of the transgene/genomic insertion region, in plants and seeds and the like, from a maize plant designated HERCULEX comprising Cry1F event TC1507. DNA sequences are provided that comprise at least one transgene/genomic insertion region junction sequence provided herein in SEQ ID NO:1, SEQ ID NO:2, SEQ ID NO:3, segments thereof, and complements of the exemplified sequences and any segments thereof. The insertion region junction sequence spans the junction between heterologous DNA inserted into the genome and the DNA from the maize cell flanking the insertion site. Such sequences are diagnostic for the subject event.

Based on these insert and border sequences, event-specific primers were generated. Taqman PCR analysis of the subject invention demonstrated that maize event TC1507 can be identified in different maize lines and genotypes by analysis of the PCR amplicons generated with these event-specific primer sets. These and other related procedures can be used to uniquely identify these maize lines.

In some embodiments, DNA sequences that comprise (or are complementary, at least in part) to a contiguous portion/segment of the transgene/genomic insertion regions are an aspect of this invention. Included are DNA sequences that comprise a sufficient length of polynucleotides of transgene insert sequence and a sufficient length of polynucleotides of maize genomic sequence from one or more of the subject maize plants.

Related embodiments pertain to DNA sequences that comprise at least 2, 3, 4, 5, 6, 7, 8, 9, 10, 11, 12, 13, 14, 15, 16, 17, 18, 19, 20, 21, 22, 23, 24, 25, or more contiguous nucleotides of a transgene portion of a DNA sequence of SEQ ID NO:3, or complements thereof, and a similar length of flanking maize DNA sequence selected from the group consisting of SEQ ID NO:1 and SEQ ID NO:2, or complements thereof. Such sequences are useful as, for example, DNA primers in DNA amplification methods. Components of the invention also include the amplicons produced by such DNA primers and homologous primers.

This invention also includes methods of detecting the presence of DNA, in a sample, from at least one of the maize plants referred to herein. Such methods can comprise: (a) contacting the sample comprising DNA with a primer set that, when used in a nucleic acid amplification reaction, of the subject invention, with DNA from at least one of these maize events; (b) performing a TAQMAN PCR amplification reaction using a reference gene identified herein; and (c) analyzing the results.

In still further embodiments, the subject invention includes methods of producing a maize plant comprising a cry1F event of the subject invention, wherein said method comprises the steps of: (a) sexually crossing a first parental maize line (comprising an expression cassettes of the present invention, which confers said insect resistance trait to plants of said line) and a second parental maize line (that lacks this insect tolerance trait) thereby producing a plurality of progeny plants; and (b) selecting a progeny plant based on results of at least one assay technique of the subject invention. Such methods may optionally comprise the further step of back-crossing the progeny plant to the second parental maize line to producing a true-breeding maize plant that comprises said insect tolerance trait. According to another aspect of the invention, related methods of determining the zygosity of progeny of a cross are provided.

DNA detection kits can be developed using the compositions disclosed herein and methods well known in the art of DNA detection. The kits are useful for identification of the subject maize event DNA in a sample and can be applied to methods for breeding maize plants containing this DNA. The kits contain DNA sequences homologous or complementary to the amplicons, for example, disclosed herein, or to DNA sequences homologous or complementary to DNA contained in the transgene genetic elements of the subject events. These DNA sequences can be used in DNA amplification reactions or as probes in a DNA hybridization method. The kits may also contain the reagents and materials necessary for the performance of the detection method.

A “probe” is an isolated nucleic acid molecule to which is attached a conventional detectable label or reporter molecule (such as a radioactive isotope, ligand, chemiluminescent agent, or enzyme). Such a probe is complementary to a strand of a target nucleic acid, in the case of the present invention, to a strand of genomic DNA from one of said maize events, whether from a maize plant or from a sample that includes DNA from the event. Probes according to the present invention include not only deoxyribonucleic or ribonucleic acids but also polyamides and other probe materials that bind specifically to a target DNA sequence and can be used to detect the presence of that target DNA sequence.

“Primers” are isolated nucleic acids that are annealed to a complementary target DNA strand by nucleic acid hybridization to form a hybrid between the primer and the target DNA strand, and can be used in conjunction with a polymerase, e.g., a DNA polymerase. Primer pairs of the present invention refer to their use for amplification of a target nucleic acid sequence, e.g., by the polymerase chain reaction (PCR) or other conventional nucleic-acid amplification methods.

Probes and primers (and amplicons) are generally 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, or 500 polynucleotides or more in length. Such probes and primers hybridize specifically to a target sequence under high stringency hybridization conditions. Preferably, probes and primers according to the present invention have complete sequence similarity with the target sequence, although probes differing from the target sequence and that retain the ability to hybridize to target sequences may be designed by conventional methods.

Methods for preparing and using probes and primers are described, for example, in Molecular Cloning: A Laboratory Manual, 2nd ed., vol. 1-3, ed. Sambrook et al., Cold Spring Harbor Laboratory Press, Cold Spring Harbor, N.Y., 1989. PCR-primer pairs can be derived from a known sequence, for example, by using computer programs intended for that purpose.

Primers and probes based on the flanking DNA and insert sequences disclosed herein can be used to confirm (and, if necessary, to correct) the disclosed sequences by conventional methods, e.g., by re-cloning and sequencing such sequences.

The nucleic acid probes and primers of the present invention hybridize under stringent conditions to a target DNA sequence. Any conventional nucleic acid hybridization or amplification method can be used to identify the presence of DNA from a transgenic event in a sample. Nucleic acid molecules or fragments thereof are capable of specifically hybridizing to other nucleic acid molecules under certain circumstances. As used herein, two nucleic acid molecules are said to be capable of specifically hybridizing to one another if the two molecules are capable of forming an anti-parallel, double-stranded nucleic acid structure. A nucleic acid molecule is said to be the “complement” of another nucleic acid molecule if they exhibit complete complementarity. As used herein, molecules are said to exhibit “complete complementarity” when every nucleotide of one of the molecules is complementary to a nucleotide of the other. Two molecules are said to be “minimally complementary” if they can hybridize to one another with sufficient stability to permit them to remain annealed to one another under at least conventional “low-stringency” conditions. Similarly, the molecules are said to be “complementary” if they can hybridize to one another with sufficient stability to permit them to remain annealed to one another under conventional “high-stringency” conditions. Conventional stringency conditions are described by Sambrook et al., 1989. Departures from complete complementarity are therefore permissible, as long as such departures do not completely preclude the capacity of the molecules to form a double-stranded structure. In order for a nucleic acid molecule to serve as a primer or probe it need only be sufficiently complementary in sequence to be able to form a stable double-stranded structure under the particular solvent and salt concentrations employed.

As used herein, a substantially homologous sequence is a nucleic acid sequence that will specifically hybridize to the complement of the nucleic acid sequence to which it is being compared under high stringency conditions. The term “stringent conditions” is functionally defined with regard to the hybridization of a nucleic-acid probe to a target nucleic acid (i.e., to a particular nucleic-acid sequence of interest) by the specific hybridization procedure discussed in Sambrook et al., 1989, at 9.52-9.55. See also, Sambrook et al., 1989 at 9.47-9.52 and 9.56-9.58. Accordingly, the nucleotide sequences of the invention may be used for their ability to selectively form duplex molecules with complementary stretches of DNA fragments.

Depending on the application envisioned, one can use varying conditions of hybridization to achieve varying degrees of selectivity of probe towards target sequence. For applications requiring high selectivity, one will typically employ relatively stringent conditions to form the hybrids, e.g., one will select relatively low salt and/or high temperature conditions, such as provided by about 0.02 M to about 0.15 M NaCl at temperatures of about 50° C. to about 70° C. Stringent conditions, for example, could involve washing the hybridization filter at least twice with high-stringency wash buffer (0.233 SSC, 0.1% SDS, 65° C.). Appropriate stringency conditions which promote DNA hybridization, for example, 6.0×sodium chloride/sodium citrate (SSC) at about 45° C., followed by a wash of 2.0×SSC at 50° C. are known to those skilled in the art, 6.3.1-6.3.6. For example, the salt concentration in the wash step can be selected from a low stringency of about 2.0×SSC at 50° C. to a high stringency of about 0.2×SSC at 50° C. In addition, the temperature in the wash step can be increased from low stringency conditions at room temperature, about 22° C., to high stringency conditions at about 65° C. Both temperature and salt may be varied, or either the temperature or the salt concentration may be held constant while the other variable is changed. Such selective conditions tolerate little, if any, mismatch between the probe and the template or target strand. Detection of DNA sequences via hybridization is well-known to those of skill in the art, and the teachings of U.S. Pat. Nos. 4,965,188 and 5,176,995 are exemplary of the methods of hybridization analyses.

In a particularly preferred embodiment, a nucleic acid of the present invention will specifically hybridize to one or more of the primers (or amplicons or other sequences) exemplified or suggested herein, including complements and fragments thereof, under high stringency conditions. In one aspect of the present invention, a nucleic acid molecule of the present invention has the nucleic acid sequence set forth in SEQ ID NOs:4-21, or complements and/or fragments thereof.

In another aspect of the present invention, a marker nucleic acid molecule of the present invention shares between 80% and 100% or 90% and 100% sequence identity with such nucleic acid sequences. In a further aspect of the present invention, a nucleic acid molecule of the present invention shares between 95% and 100% sequence identity with such sequence. Such sequences may be used in plant breeding methods, for example, to identify the progeny of genetic crosses. The hybridization of the probe to the target DNA molecule can be detected by any number of methods known to those skilled in the art, these can include, but are not limited to, fluorescent tags, radioactive tags, antibody based tags, and chemiluminescent tags.

Regarding the amplification of a target nucleic acid sequence (e.g., by PCR) using a particular amplification primer pair, “stringent conditions” are conditions that permit the primer pair to hybridize only to the target nucleic-acid sequence to which a primer having the corresponding wild-type sequence (or its complement) would bind and preferably to produce a unique amplification product, the amplicon.

The term “specific for (a target sequence)” indicates that a probe or primer hybridizes under stringent hybridization conditions only to the target sequence in a sample comprising the target sequence.

As used herein, “amplified DNA” or “amplicon” refers to the product of nucleic-acid amplification of a target nucleic acid sequence that is part of a nucleic acid template. For example, to determine whether the maize plant resulting from a sexual cross contains transgenic event genomic DNA from the maize plant of the present invention, DNA extracted from a maize plant tissue sample may be subjected to nucleic acid amplification method using a primer pair that includes a primer derived from flanking sequence in the genome of the plant adjacent to the insertion site of inserted heterologous DNA, and a second primer derived from the inserted heterologous DNA to produce an amplicon that is diagnostic for the presence of the event DNA. The amplicon is of a length and has a sequence that is also diagnostic for the event. The amplicon may range in length from the combined length of the primer pairs plus one nucleotide base pair, and/or the combined length of the primer pairs plus about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, or 500, 750, 1000, 1250, 1500, 1750, 2000, or more nucleotide base pairs (plus or minus any of the increments listed above). Alternatively, a primer pair can be derived from flanking sequence on both sides of the inserted DNA so as to produce an amplicon that includes the entire insert nucleotide sequence. A member of a primer pair derived from the plant genomic sequence may be located a distance from the inserted DNA sequence. This distance can range from one nucleotide base pair up to about twenty thousand nucleotide base pairs. The use of the term “amplicon” specifically excludes primer dimers that may be formed in the DNA thermal amplification reaction.

Nucleic-acid amplification can be accomplished by any of the various nucleic-acid amplification methods known in the art, including the polymerase chain reaction (PCR). A variety of amplification methods are known in the art and are described, inter alia, in U.S. Pat. No. 4,683,195 and U.S. Pat. No. 4,683,202. PCR amplification methods have been developed to amplify up to 22 kb of genomic DNA. These methods as well as other methods known in the art of DNA amplification may be used in the practice of the present invention. The sequence of the heterologous transgene DNA insert or flanking genomic sequence from a subject maize event can be verified (and corrected if necessary) by amplifying such sequences from the event using primers derived from the sequences provided herein followed by standard DNA sequencing of the PCR amplicon or of the cloned DNA.

The amplicon produced by these methods may be detected by a plurality of techniques. Agarose gel electrophoresis and staining with ethidium bromide is a common well known method of detecting DNA amplicons. Another such method is Genetic Bit Analysis where an DNA oligonucleotide is designed which overlaps both the adjacent flanking genomic DNA sequence and the inserted DNA sequence. The oligonucleotide is immobilized in wells of a microwell plate. Following PCR of the region of interest (using one primer in the inserted sequence and one in the adjacent flanking genomic sequence), a single-stranded PCR product can be hybridized to the immobilized oligonucleotide and serve as a template for a single base extension reaction using a DNA polymerase and labelled ddNTPs specific for the expected next base. Readout may be fluorescent or ELISA-based. A signal indicates presence of the insert/flanking sequence due to successful amplification, hybridization, and single base extension.

All patents, patent applications, provisional applications, and publications referred to or cited herein are incorporated by reference in their entirety to the extent they are not inconsistent with the explicit teachings of this specification.

### EXAMPLES

### Example 1

### Isolation of Total Genomic DNA and Quantification and PCR Primer Amplification

The isolation of genomic DNA from Cry1F homozygotes, hemizygotes and wild type samples was isolated from the individual samples by punching eight leaf discs per sample, and grinding the discs to a fine powder using a Genogrinder 2000. DNA was extracted using customized ChargeSwitch® gDNA plant kits (Invitrogen, Carlsbad, Calif.) or the Qiagen DNeasy 96-well kits (Valencia, Calif.). Prior to PCR, DNA samples were quantified with Quant-iT™ PicoGreen® Quantification Kit (Invitrogen, Carlsbad, Calif.) using manufacturer's instructions.

Oligonucleotide primer and dual labeled TaqMan probes with FAM and black hole quencher 1 (BHQ1) were synthesized by MWG Biotech (High Point, N.C.) (Table 1b).

TABLE 1b
Sequences of the Primers and Dual-Labeled TaqMan Probes

| | | | | | SEQ | PCR |
|---|---|---|---|---|---|---|
| | accession | oligo | original | | ID | product |
| gene | no. | name | name | sequence | NO: | length |
| Cry1F | | TC1507-F | MaiY-F1 | 5′-TAGTCTTCGGCCAGAATGG-3′ | 4 | 58 |
| | | TC1507-R | MaiY-R3 | 5′-CTTTGCCAAGATCAAGCG-3′ | 5 | |
| | | TC1507-P | MaiY-S1 | 5′-FAM-TAACTCAAGGCCCTCACTCCG-BHQ1-3′ | 6 | |
| ivr | U16123 | ivr104-F | INV104-F | 5′CGCTCTGTACAAGCGTGC3′ | 7 | 104 |
| | | ivr104-R | INV104-R | 5′GCAAAGTGTTGTGCTTGGACC3′ | 8 | |
| | | ivr104-P | INV104- | 5′Cy5-CACGTGAGAATTTCCGTCTACTCGAGCCT-BHQ2-3′ | 9 | |
| | | | probe | | | |
| ivr | U16123 | ivr-F | | 5′TGGCGGACGACGACTTGT3′ | 10 | 79 |
| | | ivr-R | | 5′AAAGTTTGGAGGCTGCCGT3′ | 11 | |
| | | ivr-P | | 5′-Cy5-CGAGCAGACCGCCGTGTACTTCTACC-BHQ2-3′ | 12 | |
| adh1 | X04050 | adh-F | | 5′CGTCGTTTCCCATCTCTTCCTCC3′ | 13 | 136 |
| | | adh-R | | 5′CCACTCCGAGACCCTCAGTC3′ | 14 | |
| | | adh-P | | 5′-Cy5-AATCAGGGCTCATTTTCTCGCTCCTCA-BHQ23′ | 15 | |
| hmga | AJ131373 | hmg-F | | 5′TTGGACTAGAAATCTCGTGCTGA3′ | 16 | 79 |
| | | hmg-R | | 5′GCTACATAGGGAGCCTTGTCCT3′ | 17 | |
| | | hmg-P | | 5′-Cy5-CAATCCACACAAACGCACGCGTA-BHQ2-3′ | 18 | |
| zein | X07535 | zein-F | | 5′TGCAGCAACTGTTGGCCTTA3′ | 19 | 72 |
| | | zein-R | | 5′TCATGTTAGGCGTCATCATCTGT3′ | 20 | |
| | | zein-P | | 5′-Cy5-CATCACTGGCATCGTCTGAAGCGG-BHQ2-3′ | 21 | |

Dual labeled TaqMan probes with Cy5 and BHQ2 were synthesized by IDT (Integrated DNA Technologies, Coralville, Iowa). All primers were dissolved in 1× Tris-EDTA to 200 μM and probes to 100 μM. Working stocks of the primers and dual labeled TaqMan probes were 10—fold-diluted with molecular grade water.

PCR reactions were set up in accordance with Table 2a, 2b and 2c for mono-plex reactions, using concentrations of MgCl₂ from 2.5 mM to 5.5 mM.

TABLE 2a
PCR mixture for each reaction with
25 μl final volume (2.5 mM MgCl₂).

| Component | Volume (μl) |
|---|---|
| Water | 16.85 |
| 10XPCR buffer (with 15 mMgCl₂) | 2.5 |
| 25 mM MgCl₂ | 1 |
| 10 mM dNTP (2.5 mM each) | 0.75 |
| Forward primer - 20 μM | 0.25 |
| Reverse primer - 20 μM | 0.25 |
| Dual-labeled Probe - 10 μM | 0.2 |
| HotStarTaq (5 U/μl) | 0.2 |
| Genomic DNA template (10 ng/μl) | 3 |
| Total reaction volume | 25.00 |

TABLE 2b
PCR mixture for each reaction with
25 μl final volume (4 mM MgCl₂).

| Component | Volume (μl) |
|---|---|
| Water | 15.85 |
| 10XPCR buffer (with 15 mMgCl₂) | 2.5 |
| 25 mM MgCl₂ | 2 |
| 10 mM dNTP (2.5 mM each) | 0.75 |
| Forward primer - 20 μM | 0.25 |
| Reverse primer - 20 μM | 0.25 |
| Dual-labeled Probe - 10 μM | 0.2 |
| HotStarTaq (5 U/μl) | 0.2 |
| Genomic DNA template (10 ng/μl) | 3 |
| Total reaction volume | 25.00 |

TABLE 2c
PCR mixture for each reaction with
25 μl final volume (5.5 mM MgCl₂).

| Component | Volume (μl) |
|---|---|
| Water | 13.85 |
| 10XPCR buffer (with 15 mMgCl₂) | 2.5 |
| 25 mM MgCl₂ | 4 |
| 10 mM dNTP (2.5 mM each) | 0.75 |
| Forward primer - 20 μM | 0.25 |
| Reverse primer - 20 μM | 0.25 |
| Dual-labeled Probe - 10 μM | 0.2 |
| HotStarTaq (5 U/μl) | 0.2 |
| Genomic DNA template (10 ng/μl) | 3 |
| Total reaction volume | 25.00 |

PCR reactions for multiplex reactions were set up in accordance with Table 3. HotStar Taq DNA Polymerase (HotStar Taq, 10× PCR Buffer, and 25 mM MgCl₂) from Qiagen (Valencia, Calif., Catalog #203203 or 203205) and 10 mM dNTP Nucleotide Mix from Applied Biosystems (Foster City, Calif., Catalog #N8080260) was used. Real-time PCR reactions were performed on an iCycler optical system (BioRad, Hercules, Calif.) starting with 15 minutes of denaturing at 95° C. as recommended, followed by 50 cycles of 95° C. for 15 seconds, 60° C. for 1 minute. Fluorescence signals were recorded at the end of each cycle.

Endpoint TaqMan PCR assays were set up according to Table 3. ABI GeneAmp® PCR System 9700 (Applied Biosystems, Foster City, Calif.) was used for amplification. PCR products were measured either by 4% E-Gel (Invitrogen, Carlsbad, Calif.) or by a spectrofluorometer (Tecan GENios, Männedorf, Switzerland) after an optimal number of cycles deteremined to be 28 cycles (Table 4).

TABLE 3
PCR mixture for each biplex reaction with 25 μl final volume.

| Component | Volume (μl) |
|---|---|
| Water | 13.15 |
| 10XPCR buffer (with 15 mMgCl₂) | 2.5 |
| 25 mM MgCl₂ | 4 |
| 10 mM dNTP (2.5 mM each) | 0.75 |
| TC1507 Forward primer - 20 μM | 0.25 |
| TC1507 Reverse primer - 20 μM | 0.25 |
| TC1507 Dual-labeled Probe - 10 μM | 0.2 |
| Endogenous Forward primer - 20 μM | 0.25 |
| Endogenous Reverse primer - 20 μM | 0.25 |
| Endogenous Dual-labeled Probe - 10 μM | 0.2 |
| HotStarTaq (5 U/μl) | 0.2 |
| Genomic DNA template | 3 |
| Total reaction volume | 25.00 |

TABLE 4
Instrument Settings with recommended wavelengths
for reading the PCR products.

| Dye | Excitation (nm) | Emission (nm) |
|---|---|---|
| FAM | 485 | 535 |
| Cy5 | 612 | 670 |

The real-time PCR threshold was calculated automatically by the iCycler software (version 3.0a), having a fluorescence value slightly above the background. The threshold cycle (Ct value) was determined by the number of cycles needed to generate fluorescence above the established threshold. PCR efficiencies were estimated based on the input genomic DNA and the Ct values.

The ratios of signal over background of FAM vs. Cy5 was calculated. Absolute values of the ratios were plotted for each population in Excel. Genotype calls were based on the controls (homozygotes, hemizygotes and wildtype) as well as the cluster distributions of segregating populations.

### Example 2

### PCR Efficiency Test for Maize Endogenous Genes

One aspect in developing an endpoint TaqMan zygosity assay was the selection of the most suitable endogenous gene as a reference gene. We selected invertase, a suitable reference gene, that is species-specific and has a low copy number in the genome. Four maize endogenous genes, alcohol dehydrogenase 1 (adh1), high-mobility group protein a (hmga), invertase (ivr), and zein (zein), were initially investigated out of the thousands of possibilities, as a possible reference genes for maize Cry1F in event TC1507.

The process of selecting a suitable reference gene involved first pooling 30 ng of extracted Cry1F homozygotes, hemizygotes and wild type maize genomic DNA controls (extracted according the isolation procedure described in this Example) in order to estimate the PCR efficiency for all the primers. PCR for TC1507 and the five initially selected reference genes (ivr, ivr104, adh, hmg, zein) was set up according to Table 2c. PCR products after 32 cycles were then visualized on 4% E-gel. All primers amplified expected size fragments and four reference genes had bands with similar intensities. The assay for reference gene option adh produced less product as compared to the other initially selected reference gene assays. The TC1507 event specific oligos only produced amplicons in the homozygote and hemizygote controls, with brighter band for the homozygote samples.

For the endpoint TaqMan assay, both the transgene and reference gene reactions were amplified in a single reaction (multiplexed). Attempting to achieve optimal PCR efficiencies for both genes, all primers were tested in triplicate with varied concentrations of MgCl₂ on 30 ng of genomic DNA from the homozygote samples using real-time PCR (see Table 2a, 2b and 2c).

Table 5 provides the Ct values of real-time PCR for ivr, adh, ivr104, hmg, zein and TC1507 with 30 ng of Cry1F homozygotes genomic DNA at different concentrations of MgCl₂.

TABLE 5

| MgCl₂ | ivr | adh | ivr104 | hmg | Zein | TC1507 |
|---|---|---|---|---|---|---|
| 2.5 mM | 25.6 | 26.2 | 25.7 | 23.95 | 25.4 | 21.75 |
| 4 mM | 25.85 | 28.45 | 24.95 | 23.3 | 20.9 | 21.15 |
| 5.5 mM | 25.15 | 26.15 | 24.7 | 23.2 | 20.65 | 20.65 |

The mean values of the cycles threshold (Ct) for the initially selected reference gene zein and TC1507 were similar (about 21) at high concentration of MgCl₂ (4 mM and 5.5 mM), while the mean values for the initially selected reference genes ivr and hmg had higher Ct values (from ˜23 to ˜25). The initially selected reference gene adh had Ct values more than 26 and was eliminated as an option. The MgCl2 concentration 5.5 mM had the lowest Ct value for the amplification reaction of all the tested genes and was thus used in subsequent experiments.

Primers for the initially selected reference genes ivr, ivr104, hmg and zein were multiplexed with TC1507 using real-time PCR, according to Table 3, with a 1:2 serial dilution of DNA from pooled homozygotes (performed in triplicate).

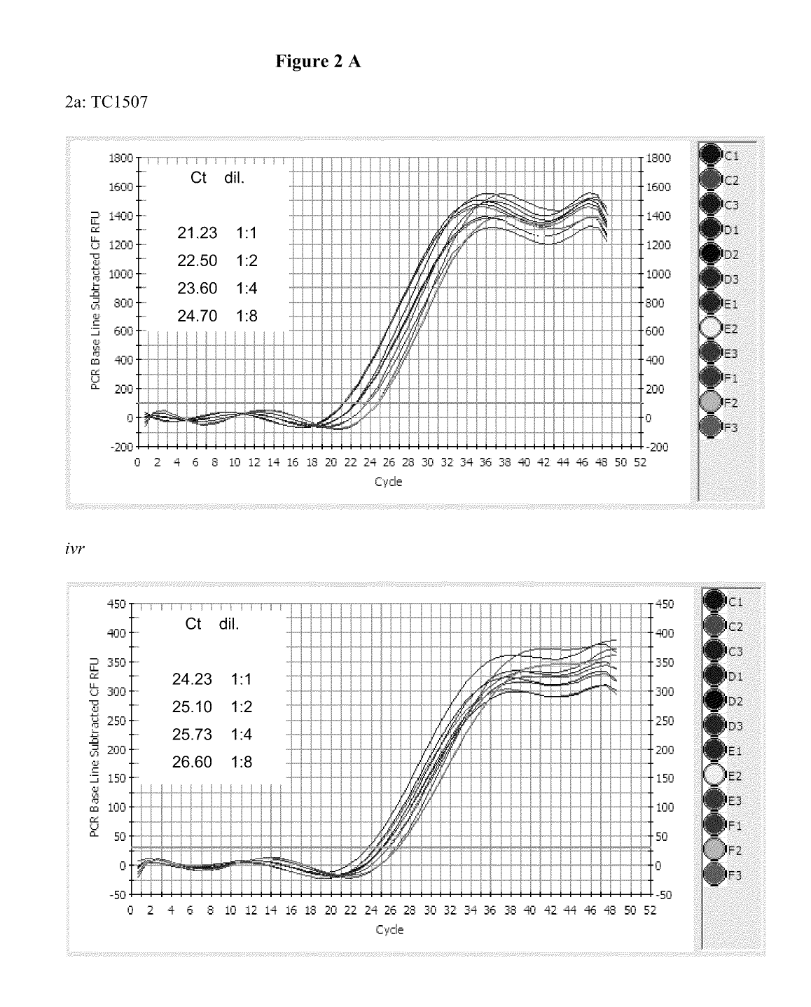

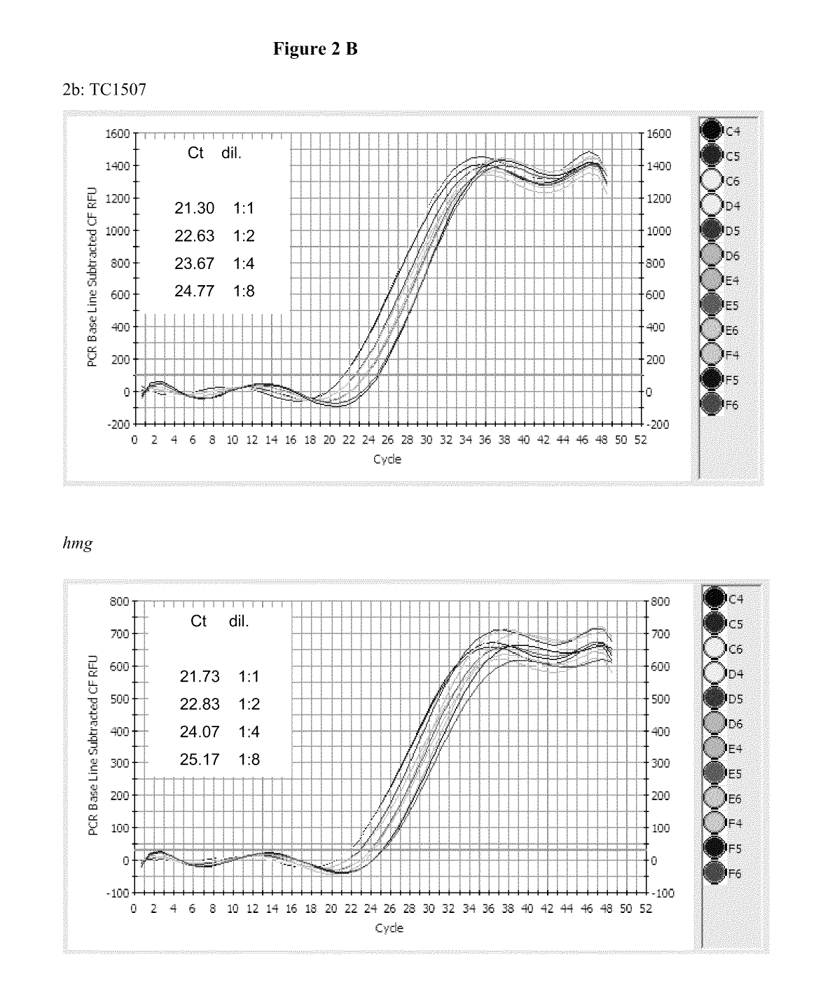

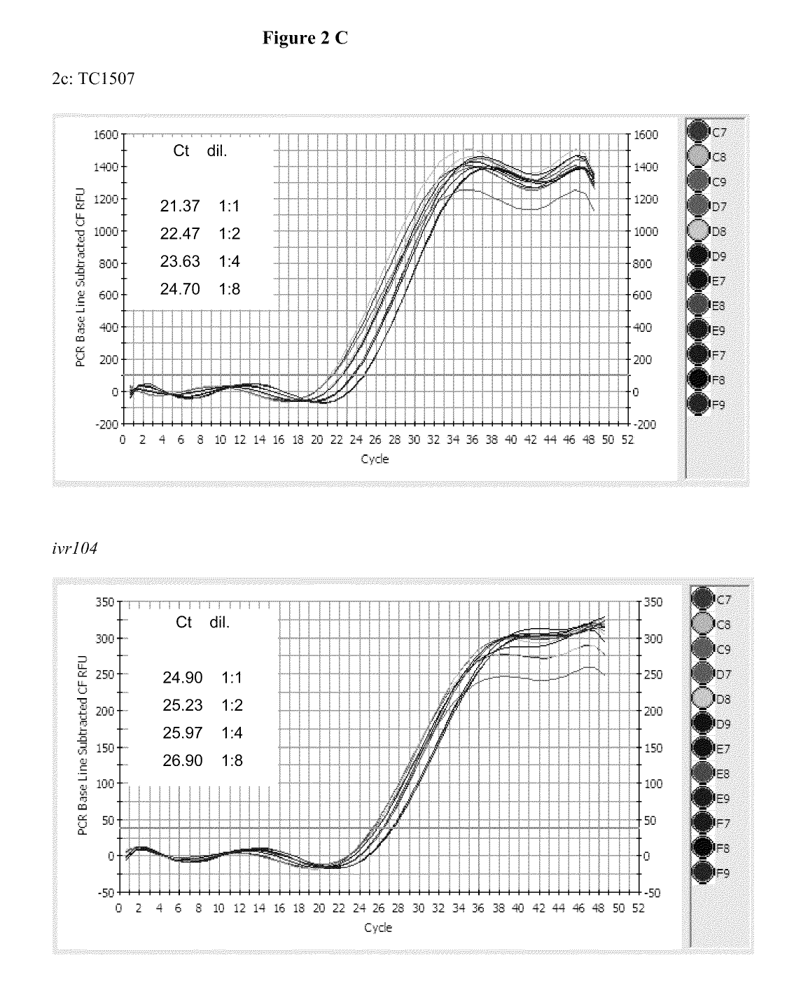

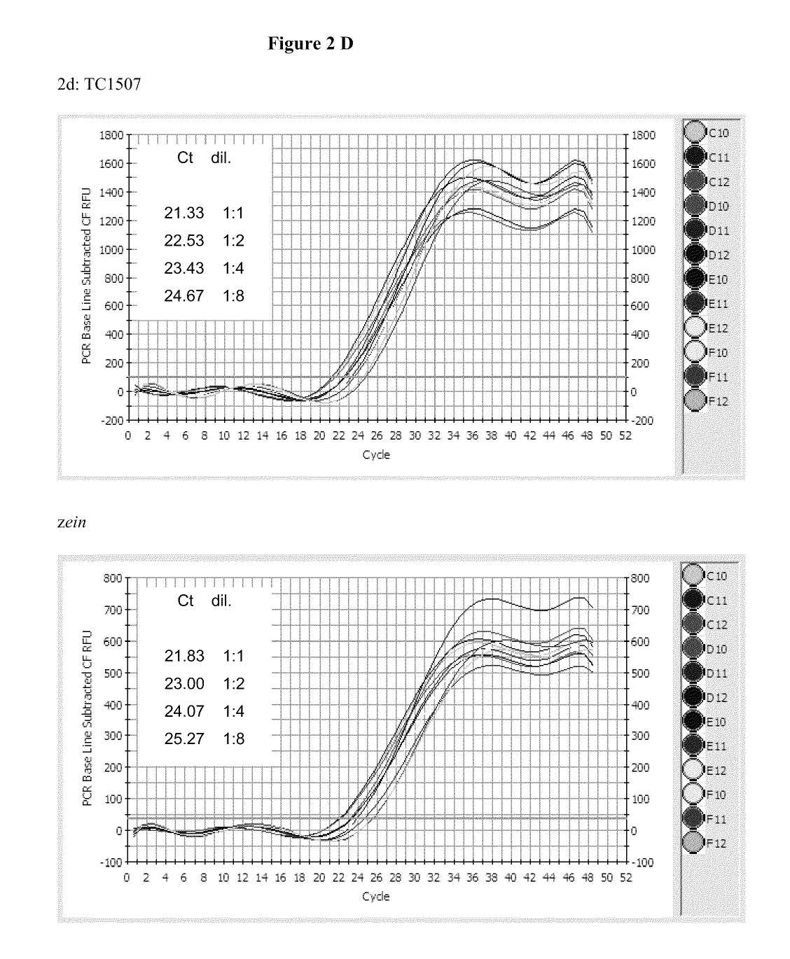

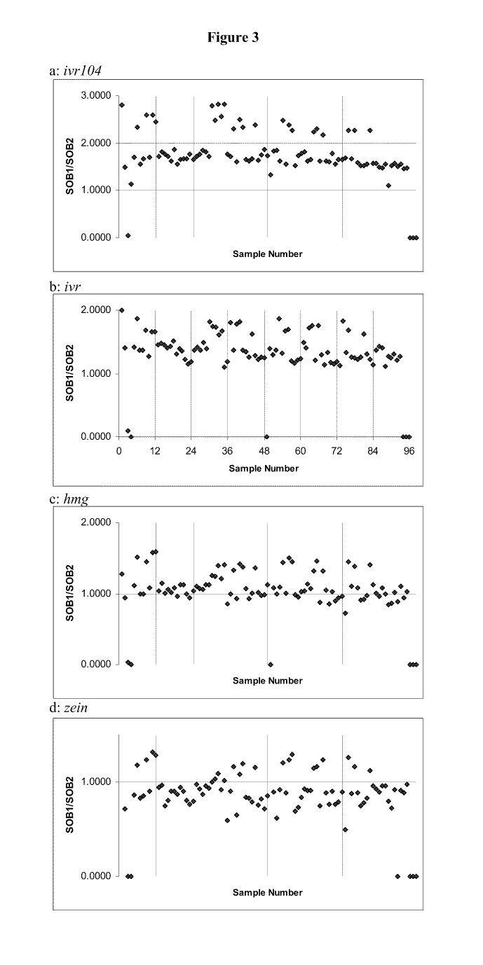

Ct values were again used to compare the efficiencies of PCR. FIG. 2 shows biplex combinations of TC1507 with different reference genes investigated. The Ct values of TC1507 demonstrated approximately one cycle difference between each dilution. The PCR efficiency for TC1507 was 100%. Reference genes performed as well as the TC1507 reactions in most dilutions.

### Example 3

### Test of Endpoint TaqMan Assay for Zygosity Genotyping

A Cry1F single stack population (Q:07K:PF04DS_ZYGO), with segregating TC1507, was used to test the multiplexing of one reference gene (ivr, ivr104, hmg or zein) with TC1507 using endpoint TaqMan PCR (Table 3). Prior to the endpoint TaqMand PCR, DNA was normalized to 10 ng/μl. The TaqMan PCR reactions were terminated at 28 cycles and then measured with a spectrofluorometer. Fluorescence signals of FAM (TC1507) over background (H₂O), as signal over background 1 (SOB1), and Cy5 (reference gene) over background 2 (SOB2) were calculated. The ratios of SOB1/SOB2 were plotted as a scatter plot in Excel. In a segregating population, three clusters of data points should be obtained allowing the cut-off points to be visually determined. It was discovered that only Ivr104 multiplexed with TC1507 under the reaction conditions disclosed herein, could make unambiguous genotypic calls. The other intially selected reference gene reactions (ivr, hmg and zein) failed to produce enough separation between homozygotes and hemizygotes to make unambiguous genotypic calls.

### Example 4

### Use of Protocol with Different Populations

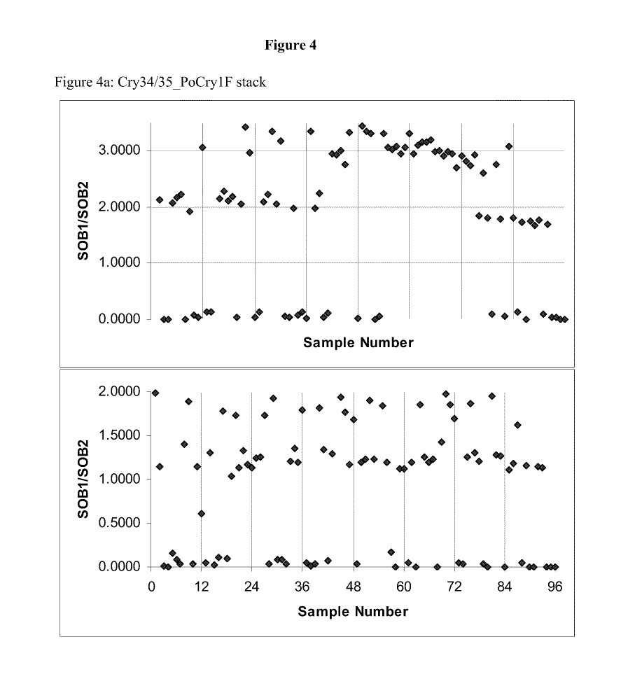

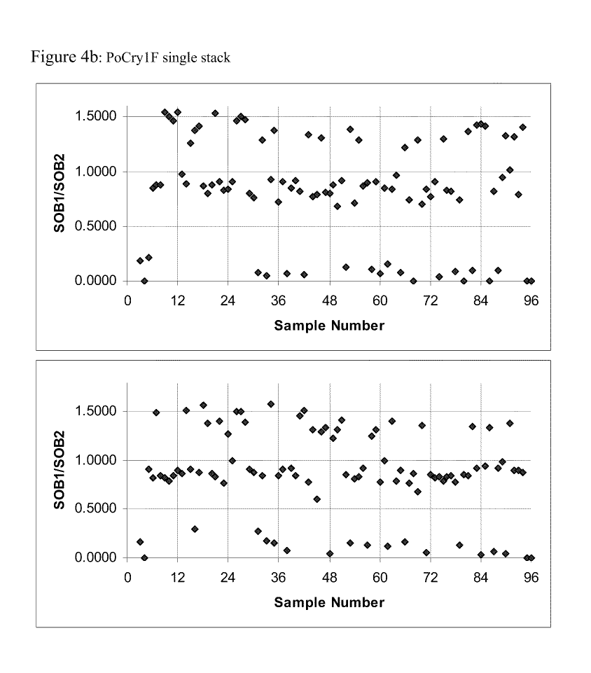

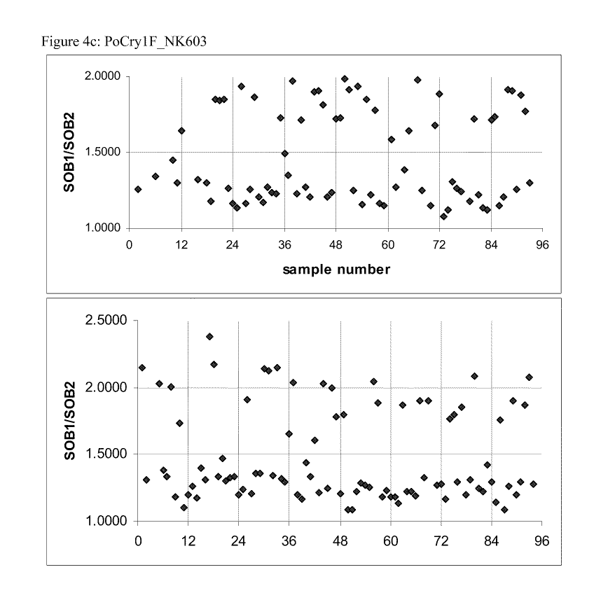

It is known that invader and PCR-based zygosity analysis (5) can be affected by the genetic background of plants. The endpoint TaqMan zygosity assay for Herculex I event TC1507 was tested for effect by the genetic background of plants with three populations, from different genetic backgrounds. Each background consisted of 184 samples (two 96-well plates of DNA). The three populations were: Cry34/35_PoCry1F and PoCry1F_NK603 double stacks and PoCry1F single stack. As illustrated in FIG. 4a and FIG. 4b, both Cry34/35_PoCry1F and PoCry1F produced the typical three clusters with homozygotes on the top, hemizygotes in the middle and wild type (WT) at the bottom. While PoCry1F_NK603 (FIG. 4c) had only two clusters (homozygotes and hemizygotes) since the WT plants, as expected, did not survive the herbicide spray. Comparing the results to the Invader assays, 98.8% of the scores are the same between the two analyses. In seven plants with discrepancies in the scores, 6 homozygotes in the Invader assay became hemizygotes when analyzed using endpoint TaqMan. One hemizygote became a homozygote.

A robust zygosity assay requires two alleles of a gene of interest to be clearly distinguished in a segregating population. As described in this study, different reference genes can also contribute to significant differences in results. Further, genotype calls should be based on the clusters from each population data.

It was determined that the maize endogenous gene Invertase was a suitable reference gene for the TC1507 event in corn. As such, a high throughput biplex endpoint TaqMan PCR for TC1507 event specific zygosity analysis, capable of producing robust genotype was developed according to Example 5.

### Example 5

### Use of Invertase in High Throughpout Biplex Endpoint TaqMan PCR to Determine Zygosity of Herculex® I Event TC1507 in Corn

One typically establishes PCR and thermal cycling conditions that amplify both transgene and/or reference sequences in a known genomic DNA template with acceptable relative fluorescence units (RFU). If the endogenous reference gene is not amplified or if the transgene sequences are not amplified at the fluorescence readings 0.5-1 unit higher than the transgenic control, optimization by varying the primer concentration and/or other parameters can be conducted.

Template DNA: Eight leaf discs per sample were sampled, and template DNA was prepared according to manufacturer's instructions (Genomic DNA extraction kit (DNeasy 96-well kit, Qiagen, Valencia, Calif., Catalog #69181) or equivalent). (A further description of some additional materials and their sources can be found in Example 1.) In general, 30 ng of total genomic DNA per 25 μl reaction yielded the best results.

Test and Control Substances: Negative control corn DNA samples were non-transgenic or transgenic corn leaf DNA not containing Herculex® I Event TC1507.

Herculex® I Event TC1507 corn DNA samples were transgenic corn leaf DNA samples containing Herculex® I Event TC1507 that were either hemizygous or homozygous. A hemizygous sample can be made if one is unavailable by combining equal proportions of negative control DNA to homozygous Herculex® I corn DNA.

Positive and negative controls are illustrated in Table 6.

TABLE 6

| Type of Control | Description | Expected Result | Interpretation |
|---|---|---|---|
| Master mix negative | No DNA is added to | Background RFU | Mix is not contaminated. |
| control | the reaction. | readings. No PCR | |
| | | products | |
| Homozygous DNA | Genomic DNA sample | RFU readings of | Control shows |
| positive control | known to be | FAM are at least 1 | amplification of the |
| (Herculex ® I Event | homozygous for the | unit higher than that | trangene (FAM) and the |
| TC1507) | transgenic sequence is | of the non-transgenic | endogenous reference |
| | added. | control. Readings of | gene (Cy5) alleles from |
| | | Cy5 similar to non- | genomic DNA. |
| | | transgenic control. | |
| Hemizygous DNA | Genomic DNA sample | RFU readings of | Control shows |
| positive control | known to be | FAM are at least 0.5 | amplification of the |
| (Herculex ® I Event | hemizygous for the | unit higher than that | transgene (FAM) and the |
| TC1507) | transgenic and wild- | of the non-transgenic | endogenous reference |
| | type sequences is | control. Readings of | gene (Cy5) alleles from |
| | added. | Cy5 similar to non- | genomic DNA. |
| | | transgenic control. | |
| Non-transgenic DNA | Genomic DNA sample | Fluorescence | Control only shows |
| negative control | extracted from a non- | readings of Cy5 are at | amplification of the |
| | transgenic line of the | least 0.5-1 unit higher | endogenous reference |
| | same background as | than that of the | gene (Cy5) and not the |
| | unknowns is added. | negative background | transgene (FAM) from |
| | | control. | genomic DNA. |

Procedure for DNA Extraction, Purification, and Quantitation. The following steps were undertaking in consecutive order.
Punch 8 leaf discs per sample and transfer into Qiagen collection tubes. Clean puncher after each sampling with 70% alcohol followed by a quick rinse in water and then wipe dry.
Prepare DNA extraction buffer according to manufacturer's recommendation.
Isolate DNA following manufacturer's recommendation.
Determine the DNA concentration using Quant-iT™ PicoGreen® Quantification Kit and a spectrophotometer or equivalent.

PCR Conditions. The following steps were undertaking in consecutive order.

Prepare the following reaction mixture as a Master Mix containing all components except the DNA templates. When preparing the mixture, ensure that it is sufficient for 10% more reactions than actually required.

Following were the components for a biplex reaction containing Herculex® I Event TC1507 and endogenous gene invertase oligo nucleotides (concentrations of all DNA samples were normalized):

| Component | Volume (μl) |
|---|---|
| Water | 13.15 |
| 10XPCR buffer | 2.5 |
| 25 mM MgCl₂ | 4 |
| 10 mM dNTP (2.5 mM each) | 0.75 |
| TC1507 Forward primer - 20 μM | 0.25 |
| TC1507 Reverse primer - 20 μM | 0.25 |
| TC1507 Dual-labeled Probe - 10 μM | 0.2 |
| Invertase Forward primer - 20 μM | 0.25 |
| Invertase Reverse primer - 20 μM | 0.25 |
| Invertase Dual-labeled Probe - 10 μM | 0.2 |
| HotStarTaq (5 U/μl) | 0.2 |
| Genomic DNA template (10 ng/μl) | 3 |
| Total reaction volume | 25.00 |

Primers and Probes were prepared and utilized as follows.

TABLE 7
List of Primers and Probe Sequences.

| Primer or | |
|---|---|
| Probe Name | Sequence |
| INV104-F | 5′-CGCTCTGTACAAGCGTGC-3′ |
| (Invertase | (SEQ ID NO: 7) |
| Forward | |
| Primer) | |
| INV104-R | 5′-GCAAAGTGTTGTGCTTGGACC-3′ |
| (Invertase | (SEQ ID NO: 8) |
| Reverse | |
| Primer) | |
| INV104-probe | 5′-CY5-CACGTGAGAATTTCCGTCT |
| (Invertase | ACTCGAGCCT-BHQ2-3′ |
| labeled Probe) | (SEQ ID NO: 9) |
| MaiY-F1 | 5′-TAGTCTTCGGCCAGAATGG-3′ |
| (TC1507 | (SEQ ID NO: 4) |
| Forward | |
| Primer) | |
| MaiY-R3 | 5′-CTTTGCCAAGATCAAGCG-3′ |
| (TC1507 | (SEQ ID NO: 5) |
| Reverse | |
| Primer) | |
| MaiY-S1 | 5′-FAM-TAACTCAAGGCCCTCACT |
| (TC1507 | CCG-BHQ1-3′ |
| Dual-labeled | (SEQ ID NO: 6) |
| Probe) | |

TABLE 8
Preparation of primer stock solutions (200 μM).

| Allele | Forward | nmoles | Dilution | Reverse | nmoles | Dilution |
|---|---|---|---|---|---|---|
| Invertase | INV104-F | 33.2 | Add 166 μl of | INV104-R | 35.0 | Add 175 μl of |
| Reference gene | | | 1x Tris-EDTA | | | 1x Tris-EDTA |
| Herculex I | MaiY-F1 | 112.5 | Add 562.5 μl of | MAIY-R3 | 96.4 | Add 482 μl of |
| event - TC1507 | | | 1x Tris-EDTA | | | 1x Tris-EDTA |

Primer stocks were aliquoted and diluted 1:10 with H₂O to a final working concentration of 20 μM.

TABLE 9
Preparation of probe stock solutions (100 μM).

| Allele | Probe | nmoles | Dilution |
|---|---|---|---|
| Invertase | INV104-Probe | 17.3 | Add 173.0 μl of |
| Reference gene | | | 1x Tris-EDTA |
| Herculex I event - | MaiY-S1 | 16.3 | Add 163.0 μl of |
| TC1507 | | | 1x Tris-EDTA |

Probe stocks were aliquoted and diluted 1:10 with H₂O to a final working concentration of 10 μM. Probes are light sensitive and should be stored in dark as much as possible. Multiple aliquots of each probe should be made to minimize the number of freezing and thawing cycles.

PCR assays were set up with appropriate controls. When a 96-well plate is used, it is recommended that the following wells be used for controls: H11−H12=negative controls (reagents but no DNA), A1=homozygous positive control containing Herculex® I TC1507 corn genomic DNA; A2=hemizygous positive control containing Herculex® I Event TC1507; A3=negative control containing corn genomic DNA with no Herculex® I Event TC1507, and A4−H10=unknown samples.

DNA was amplified in a GenAmp PCR System 9700 under the following conditions:

| Cycle | Temp | | Number |
|---|---|---|---|
| Element | (° C.) | Time | of Cycles |
| Initial | 95 | 15 minutes | 1 |
| Denaturation | | | |
| Denaturation | 95 | 15 seconds | |
| Annealing & | 60 | 60 seconds | 28 |
| Extension | | | |

Samples were analyzed as follows.
Instrument Setting: recommended wavelengths for reading the PCR results are as follows.

| Dye | Excitation (nm) | Emission (nm) |
|---|---|---|
| FAM | 485 | 535 |
| (Herculex ® I TC1507) | | |
| Cy5 (Invertase) | 612 | 670 |

Samples not containing Herculex® I Event TC1507 genomic DNA will only result in the fluorescence readings of the reference gene PCR product. Samples containing hemizygous or homozygous Herculex® I Event TC1507 genomic DNA will result in RFU readings for the FAM probe at least 0.5-1 unit higher than that of the negative background control. If samples yield no PCR products for the transgene or endogenous gene alleles, DNA may not be of adequate quality or quantity. In that case, a new DNA preparation or a new reaction should be performed. Results are acceptable when:
the known hemizygous and homozygous controls show expected high fluorescence readings for the Herculex® I Event TC1507 and reference gene invertase. A reading of 0.5-1.0 unit should separate the SOB1/SOB2 ratios between the two controls.
the negative control must show very low fluorescence readings for both Herculex® I Event TC1507 and reference genes.
the non-transgenic DNA control must show the fluorescence reading for the reference gene only.

Following completion of the TaqMan PCR and fluorescence reading, a table and distribution graph were generated (Table 10, FIG. 1). The ‘wildtype’ (Wt), ‘hemizygous’, and ‘homozygous’ controls of similar genotypic background can serve as negative and positive controls. In a segregating population, three clusters of data points should be obtained allowing the cut-off points to be visually determined. These cut-off points are arbitrary and separation between clusters is usually about 0.5-1 unit. However, data points could scatter due to variability in the assay. For the example illustrated below, three clusters of data points are clearly visible. The data points for wildtype are less than 0.5, those for ‘hemizygous’ samples range from 0.5-1.1, and those for ‘homozygous’ are above 1.1.

Table 10 Example of a Data Table. Reporter1=FAM reading, Reporter232 Cy5 reading, SOB1=Signal over the background of FAM (ratio of sample signal over average of background signal at 535 nm), SOB2=Signal over background of Cy5 (Ratio of sample signal over average of background signal at 670 nm), Ratio=SOB1/SOB2 (absolute value), Call=Interpretation of wild-type (ratio<0.5), hemizygous (ratio>0.5 but<1.1), and homozygous (ratio>1.1) samples for Herculex® I Event TC1507.

TABLE 10

| Sample | Wells | Reporter1 | SOB1 | Sample# | Reporter2 | SOB2 | Ratio | Call |
|---|---|---|---|---|---|---|---|---|
| Sam1 | A5 | 10807 | −16.9 | 5 | 35670 | 114.3 | 0.1481 | Wt |
| Sam2 | A6 | 24314 | 86.9 | 6 | 33617 | 101.9 | 0.8525 | Hemi |
| Sam3 | A7 | 25918 | 99.2 | 7 | 35358 | 112.4 | 0.8829 | Hemi |
| Sam4 | A8 | 26255 | 101.8 | 8 | 35989 | 116.2 | 0.8764 | Hemi |
| Sam5 | A9 | 37931 | 191.6 | 9 | 37283 | 124.0 | 1.5455 | Hmz |
| Sam6 | A10 | 37176 | 185.8 | 10 | 37269 | 123.9 | 1.4997 | Hmz |
| Sam7 | A11 | 35059 | 169.5 | 11 | 35899 | 115.6 | 1.4657 | Hmz |
| Sam8 | A12 | 45525 | 250.0 | 12 | 43689 | 162.4 | 1.5388 | Hmz |
| Sam9 | B1 | 30329 | 133.1 | 13 | 39359 | 136.4 | 0.9759 | Hemi |
| Sam10 | B2 | 28052 | 115.6 | 14 | 38354 | 130.4 | 0.8869 | Hemi |
| Sam11 | B3 | 33754 | 159.5 | 15 | 37700 | 126.5 | 1.2610 | Hmz |
| Sam12 | B4 | 31538 | 142.4 | 16 | 33828 | 103.2 | 1.3801 | Hmz |
| Sam13 | B5 | 31319 | 140.8 | 17 | 33242 | 99.7 | 1.4120 | Hmz |
| Sam14 | B6 | 26514 | 103.8 | 18 | 36459 | 119.0 | 0.8724 | Hemi |
| Sam15 | B7 | 24997 | 92.2 | 19 | 35733 | 114.7 | 0.8038 | Hemi |
| Sam16 | B8 | 27446 | 111.0 | 20 | 37665 | 126.3 | 0.8790 | Hemi |
| Sam17 | B9 | 34671 | 166.5 | 21 | 34785 | 109.0 | 1.5284 | Hmz |
| Sam18 | B10 | 30456 | 134.1 | 22 | 41250 | 147.8 | 0.9075 | Hemi |
| Sam19 | B11 | 26476 | 103.5 | 23 | 37491 | 125.2 | 0.8268 | Hemi |
| Sam20 | B12 | 26262 | 101.9 | 24 | 36842 | 121.3 | 0.8398 | Hemi |

On completion of PCR and fluorescence readings, a distribution graph was generated as described above. See FIG. 1.

What is claimed is:

1. A method for determining zygosity of a TC1507 event in Zea mays tissue, said TC1507 event comprising a transgene construct comprising a cry1F gene, said method comprising using a fluorescence-based endpoint Taq PCR assay to detect
said TC1507 event and an endogenous reference gene
said method comprising:
obtaining a sample of genomic DNA from said Zea mays tissue,
contacting said sample with
a. an event forward primer and an event reverse primer, wherein at least one of said event primers specifically binds said transgene construct, and wherein said primers produce an event amplicon diagnostic for said event, when present in said sample
b. a reference forward primer and a reference reverse primer that produce a reference amplicon from said endogenous reference gene
c. a florescent event probe that hybridizes with said event amplicon
d. a florescent reference probe that hybridizes with said reference amplicon
quantitating said florescent event probe that hybridized to said event amplicon,
quantitating said florescent reference probe that hybridized to said reference amplicon, comparing amounts of hybridized florescent event probe to hybridized florescent reference probe; and
determining zygosity of TC1507 by comparing florescence ratios of hybridized fluorescent event probe and hybridized fluorescent reference probe.

2. The method of claim 1 wherein one event primer hybridizes to a TC1507 flanking sequence and one event primer hybridizes to a said transgene construct.

3. The method of claim 2 wherein said TC1507 flanking sequence is selected from the group consisting of SEQ ID NO:1 and SEQ ID NO:2.

4. The method of claim 2 wherein said transgene construct is residues 2830-9015 of SEQ ID NO:3.

5. The method of claim 1 wherein said reference gene is an endogenous Zea mays invertase gene.

6. The method of claim 2 wherein said flanking sequence is residues 2629-2829 of SEQ ID NO:3.

7. The method of claim 2 wherein said flanking sequence is residues 9016-9216 of SEQ ID NO:3.

8. The method of claim 5 wherein said method is used for breeding introgression of the TC1507 event into another corn line.

9. The method of claim 8, wherein said another corn line is a null TC1507 Zea mays line.

10. The method of claim 1 wherein said event amplicon is 58 basepairs.

11. The method of claim 5 wherein said reference gene comprises or hybridizes to a sequence selected from the group consisting of SEQ ID NO:7, SEQ ID NO:8, and SEQ ID NO:9.

12. The method of claim 1 wherein said reference primers comprise SEQ ID NO: 7 and SEQ ID NO:8, and said reference probe comprises SEQ ID NO:9.

13. The method of claim 1 wherein said probes are labeled with a fluorescent dye and quencher.

14. The method of claim 13 wherein said event probe comprises FAM as said fluorescent dye at the 5′ end of said event probe and a Black Hole Quencher 1 (BHQ1) as said quencher on the 3′ end of said event probe.

15. The method of claim 1 wherein said reference gene is a conserved maize endogenous gene capable of single or low copy number PCR amplification.

16. The method of claim 13 wherein said reference probe is labeled with Cy5 at the 5′ end of said reference probe and a Black Hole Quencher 2 (BHQ2) at the 3′ end of said reference probe.

17. The method of claim 12 wherein said reference amplicon is a 104 basepair fragment amplified by said primers.

18. The method of claim 1 wherein said reference probe comprises SEQ ID NO:9.

19. The method of claim 1 wherein said reference forward primer comprises SEQ ID NO:7 and said reference reverse primer comprises SEQ ID NO:8.

20. The method of claim 1 wherein results of said method are read directly in a plate reader

21. The method of 1 wherein said sample is obtained from a corn plant in a field.

22. A kit for performing the method of claim 1, said kit comprising
a. an event forward primer and an event reverse primer, wherein at least one of said event primers specifically binds said cry1F transgene construct, and wherein said primers produce an event amplicon diagnostic for said event, when present in said sample;
b. a reference forward primer and a reference reverse primer that produce a reference amplicon from said endogenous reference gene;
c. an event probe that hybridizes with said event amplicon; and
d. a reference probe that hybridizes with said reference amplicon.
